(19) United States
(12) Patent Application Publication
(10) Pub. No.: US 2012/0134928 A1
(43) Pub. Date: May 31, 2012

(54) MODULATION OF TERMINASE ACTIVITY AT TELOMERES AND DNA DOUBLE-STRAND BREAKS

(75) Inventors: Victoria Lundblad, Del Mar, CA (US); Bridget Baumgartner, San Diego, CA (US)

(73) Assignees: BAYLOR COLLEGE OF MEDICINE, Houston, TX (US); THE SALK INSTITUTE FOR BIOLOGICAL STUDIES, La Jolla, CA (US)

(21) Appl. No.: 13/320,072

(22) PCT Filed: May 13, 2010
(86) PCT No.: PCT/US2010/034796

§ 371 Date: Jan. 25, 2012

Related U.S. Application Data
(60) Provisional application No. 61/177,792, filed on May 13, 2009.

(51) Int. Cl. A61K 49/00; C12N 5/10; C12Q 1/68; C12N 1/19

(52) U.S. Cl. 424/9.2; 435/254.2; 435/325; 435/366; 435/6.1

(57) ABSTRACT

The present invention is directed to compositions and methods concerning terminase proteins. In particular, the present invention is directed to terminase proteins and their function in processing telomeres and double-strand breaks.

### CROSS-REFERENCES TO RELATED APPLICATIONS

This application claims the benefit of U.S. Provisional Application No. 61/177,792, filed May 13, 2009, the content of which is incorporated herein by reference in its entirety and for all purposes.

### STATEMENT AS TO RIGHTS TO INVENTIONS MADE UNDER FEDERALLY SPONSORED RESEARCH AND DEVELOPMENT

The present invention was supported by a grant from the National Institutes of Health (R01 GM055867) and a USAMRMC Breast Cancer Predoctoral Fellowship (DAMD 17-03-1-0596). The Government has certain rights in the invention.

### BACKGROUND OF THE INVENTION

Telomeres, which are specialized structures found at the ends of linear chromosomes, play critical roles in genome stability and cell proliferation in both normal and transformed cells. The telomere consists of tandemly repeated DNA sequences and its associated proteins. The addition of telomere sequences to chromosome termini is mainly achieved by the enzyme telomerase, a telomere-dedicated RNA-dependent DNA polymerase that adds telomeric repeats onto the G-rich single-strand terminus present at chromosome ends. In addition to telomerase, proteins bound to the chromosome end provide a “capping” function to protect these natural DNA termini from the types of activities that can occur at DNA double-strand breaks (DSBs).

All eukaryotic cells employ mechanisms to ensure the complete replication of chromosome ends, which otherwise shorten with each cell division. The primary pathway for telomere length maintenance in most organisms relies on the enzyme telomerase. Telomerase is a reverse transcriptase that elongates the 3′ terminus of the G-rich strand of the telomere, using an internal RNA subunit as the template to dictate the sequence added to chromosome ends. As was first observed in the ciliates and yeast, and later recapitulated in human cells, absence of telomerase activity leads to a progressive decline in telomere length that inhibits the proliferative capacity of cells and heralds replicative senescence. Telomerase is therefore a critical factor in maintaining long-term cell viability.

In addition to telomerase, most models of telomere replication postulate a second enzymatic activity that acts on telomeric DNA. This hypothesized activity, referred to as terminase, is a 5′ to 3′ exonuclease that processes chromosome termini, thereby generating G-rich single-strand extensions that are crucial to telomere function. This resection activity plays an essential role at telomeres, immediately following the completion of conventional DNA replication. It has long been appreciated that semi-conservative DNA replication of linear molecules generates daughter chromosomes with non-identical termini. In particular, one of these two daughter termini will have a blunt terminus which lacks the critical G-strand overhang. This blunt terminus is unable to recruit the telomere-specific proteins that protect chromosome ends. If left unprocessed, such termini are repaired as double-strand breaks, with lethal (and often oncogenic) consequences. Thus, this invokes an obligatory nuclease activity that must resect these newly synthesized blunt termini. The action of this terminase enzyme converts blunt ends into functional capped chromosome ends, thereby preventing the telomere-telomere fusions that can help promote tumorigenesis.

Telomere biology impacts several aspects of human health. Defects in subunits of the telomerase complex, as well as at least one component of the telomere shelterin complex, are responsible for several genetically inherited telomere shortening syndromes that result in early mortality due to bone marrow failure, pulmonary fibrosis and/or liver failure. During tumorigenesis, there are also two crucial telomere-related events that occur. First, when telomeres become dysfunctional (also referred to as “uncapped”), chromosome ends become subject to end-to-end fusions, with the rampant genome instability resulting from these fused chromosomes responsible for extensive chromosome aberrations. This has led to the proposal that telomere-based crisis, occurring during the early stages of tumorigenesis, is a causative factor in the early, pre-invasive stages of cancer. The terminase activity described in the preceding paragraph is a crucial component in ensuring that telomeres are fully functional (e.g. “capped”).

A subsequent telomere-related event that occurs during oncogenesis is up-regulation of telomerase, a telomere-dedicated enzyme that stabilizes and elongates telomeres, thereby endowing cancer cells with unlimited cell growth. Reactivation of telomerase occurs in 85% to 90% of tumors, and as a consequence, there has been a substantial research investment over the past decade dedicated to the development of telomerase inhibitors as potential anti-cancer therapeutics.

In parallel with the hypothesis about a telomere-dedicated nuclease, double-strand breaks (DSBs) are also subject to processing by 5′ to 3′ nuclease activity. In budding yeast, where this process has been studied extensively, DSBs are initially detected by the Mre11-Rad50-Xrs2 complex, which leads to activation of the PI3-like kinase Tel1. At this point, two different pathways can lead to repair of a DSB. The break can be fused through a process referred to as NHEJ (non-homologous end joining) or the break can be repaired by a separate pathway called homologous recombination (HR). The choice between these two pathways is dictated by whether the DSB is resected by a 5′ to 3′ nuclease, which resects one strand of the DNA to generate a 3′ overhang. The production of this single-strand overhang effectively blocks the NHEJ pathway, and instead shunts this DNA substrate into a pathway that leads to its repair by HR. Numerous lines of evidence indicate that the terminase nuclease which is responsible for resecting newly replication chromosome termini is also responsible for resection of DSBs.

The present invention relates to terminase, an enzyme involved in processing newly replicated chromosome ends and DNA double strand breaks, with subsequent consequences on telomere function and DNA repair, respectively. The invention provides methods and compositions for identifying and measuring terminase activity. The present invention cures current problems in the art and is key to further understanding mechanisms of DSB repair and manipulating telomeric processing.

### BRIEF SUMMARY OF THE INVENTION

In one aspect, a cell including a nucleic acid that encodes a terminase protein including an impaired telomeric terminase activity is provided.

In another aspect, a method for preparing a cell expressing a terminase protein with an impaired terminase activity is provided. The method includes introducing to the cell a nucleic acid encoding the terminase protein with the impaired terminase activity. The cell is allowed to express the terminase protein with the impaired terminase activity. And the impaired terminase activity in the cell is compared to a terminase activity in a control cell.

In another aspect, a method of determining whether a test compound modulates terminase activity in vitro is provided. The method includes combining a test compound, a terminase protein, and a double-stranded deoxynucleotide substrate in a single reaction mixture in vitro under conditions conducive to terminase activity. Sufficient time is allowed for the terminase protein to react with the double-stranded deoxynucleotide substrate to form a hydrolyzed double-stranded deoxynucleotide product. An amount of the hydrolyzed double-stranded deoxynucleotide product is detected and the amount is compared to a control amount of hydrolyzed double-stranded deoxynucleotide product, thereby determining whether the test compound modulates terminase activity in vitro.

In one aspect, a method of determining whether a test compound modulates terminase activity in a cell is provided. The method includes determining that the test compound modulates terminase activity in vitro according to the method provided herein. The test compound is contacted with a cell. An amount of telomeric stability in the cell is determined and the amount is compared to a control amount of telomeric stability, thereby determining whether the test compound modulates terminase activity in the cell.

In another aspect, a method of determining whether a test compound is a terminase modulating anticancer compound is provided. The method includes determining that a test compound modulates terminase activity in vitro according to the method provided herein. The test compound is administered to a cancer model organism. It is determined whether the test compound exhibits anticancer properties in the cancer model organism.

In one aspect, a kit for determining whether a test compound modulates terminase activity in vitro is provided. The kit includes a terminase protein and a double-stranded deoxynucleotide substrate.

### BRIEF DESCRIPTION OF THE DRAWINGS

FIG. 1: A schematic drawing demonstrating that the immediate product of leading strand DNA synthesis is a blunt-ended molecule that has lost the terminal G-rich single-strand telomeric 3′ extension.

FIG. 2: Comparison of the role of terminase activity in processing newly replicated telomeres and repair of double-strand breaks.

FIG. 3: Preparation of Rat1 according to Example 1. Left lane: molecular weight standards. Right lane: Rat1-CBP preparation, assay with CBP detection assay.

FIGS. 4A-B: Assays demonstrating that the S. cerevisiae Rat1 protein possesses strand-specific DNA exonuclease activity. (A): Rat1 degrades the 5′ terminus of single-stranded DNA substrates. (B): A schematic is depicted of the double strange plasmid substrate, YEplac195, digested with Stu1 to generate blunt DNA termini, with the position of the oligonucleotide probes. Rat1 degrades double-stranded DNA substrates with 5->3′ polarity, to produce a duplex molecule with a protruding 3′ single-strand extension.

FIG. 5: Anti-Rat1 peptide antibodies recognize the endogenous Rat1 protein. Lane 1: molecule weight size standards. Lane 2: Endogenous Rat1. Lane 3: Endogenous Rat1 plus pVL3271 (ADH-Rat1). Lane 4: Endogenous Rat1 plus pVL3270 (Gal4AD-Rat1). Reference molecules weights: Rat1 (115.9 kDa); Gal4AD-Rat1 (133.8 kDa).

FIG. 6: Standardization curves for detection of the MATa and PMA1 genomic regions by Q-PCR.

FIGS. 7A-B: Degree of association of the Rat1 protein at the MATa locus, following induction of a double-strand break (DSB), as measured by ChIP. (A): Comparison of the ChIP signal at the MATa locus with pre-immune versus anti-Rat1 antibodies, as well as the association of Rat1 at the PMA1 locus. (B): Comparison of Rat1 association at MATa versus PMA1, over time following induction of a DSB at the MATa locus.

FIGS. 8A-B: Association of the Rat1 protein with telomeres through the cell cycle, following release from an alpha factor arrest. (A): ChIP experiment, using anti-Rat1 antibodies (Ab116) versus pre-immune serum, monitoring association with Tel VIR by PCR through the cell cycle. (B) Monitoring three different telomeres through the cell cycle in a Rat1-TAP strain in which Rat1 was immunoprecipitated on IgG Sepharose™.

FIGS. 9A-B: (A): Viability of a panel of rat1-ts strains at a range of different temperatures. (B): Effects on protein stability of six Rat1-ts proteins when grown at 23° versus 1 to 3 hours at 36°.

FIGS. 10A-B: (A): Sequence of the wild type S. cerevisiae Rat1 protein (SEQ ID NO:1). (B): The table in (B) summarizes the results of sequence analysis of 19 rat1-ts alleles recovered from the experiment described in Example 7, including a column indicating those amino acids, which are mutant in the rat1-ts alleles and conserved in the human XRN2 protein (SEQ ID NO:2).

FIG. 11: Direct comparison of a portion of the S. cerevisiae Rat1 and H. sapiens XRN2 proteins, generated from a BLAST search, with the seven amino acids that are identical between the two proteins and which are also mutated in the rat1-ts mutant collection. The rat1-ts alleles are indicated by dashed-line boxes, and the rat1^{term} alleles are indicated by solid boxes.

FIGS. 12A-C: Phenotypic characterization of three rat1^{term} mutations. (A): Sensitivity to HU, MMS or UV, at the indicated doses, of a rat1-Δ strain with a CEN plasmid expressing either wild type RAT1, or rat1-H63E, rat1-E253E or rat1-D724K. (YVL3042), following loss of the pVL3344 [URA3 CEN RAT1] plasmid was assessed. (B): One mutation in RAT1 (rat1-D724K) resulted in a substantial elongation of telomeres, relative to wild type (FIG. 12B), indicating a defect in telomere length regulation. (C): Synthetic lethality assay identifying one mutation, rat1-E253K, which displays a severe synthetic lethality in the absence of YKU80 function. Lanes: 1: RAT1, grown fro 50 generations; 2: RAT1, grown for 75 generations; 3: rat1-D724K, grown for 25 generations; 4: rat1-D724K, grown for 50 generations; 5: rat1-D724K, grown for 75 generations; 6: RAT1, grown for 50 generations; 7: RAT1, grown for 75 generations.

FIGS. 13A-B: Two complementary experiments demonstrating that in the absence of the Rat1 protein, resection by the terminase activity at double-strand breaks is severely impaired. (A): The figure depicts monitoring of the resection following induction of a double-strand break at the HO site at the MATa locus, by monitoring resection products in RAT1 and rat1-113 strains at 23° and 34°. (B): This figure depicts monitoring of the association of the single-strand Rad51 protein at the MATa locus by ChIP, in RAT1 and rat1-102 strains at 23° and 36°.

FIG. 14: Viability of RAT1 and rat1-102 strains, in LIG4 and lig4-Δ backgrounds, plated as serial 5-fold dilutions on YPD plates incubated at the indicated temperatures.

FIGS. 15A-C: Co-immunoprecipitation experiments monitoring the association of the Rat1 protein with subunits of the telomerase complex. (A): This figure depicts immunoprecipitates using pre-immune, anti-Rat1 or anti-myc antibodies were probed to detect the Est1-(myc)₁₃ or Est2-(myc)₁₃ proteins, which were over-expressed by the constitutive ADH promoter on a high copy plasmid. The apparent reduction in size of the Est2-(myc)₁₃ protein is due to degradation at the C-terminus, resulting in removal of some number of myc epitopes. (B) and (C): Co-immunoprecipitation of Rat1 with Est1-(myc)₁₃ expressed from its genomic locus, from either asynchronous cultures (B) or comparing asynchronous cultures with cultures arrested in the G1 or G2/M phases of the cell cycle. Lanes for FIG. 15C: 1: Asynchronous culture; 2: sigma-factor arrested culture; 3: nocodozole-arrested culture.

### DETAILED DESCRIPTION OF THE INVENTION

The present invention is directed to the identification and application of the terminase activity of the yeast gene Rat1 and the human gene XRN2. The disclosure provides methods of controlling and monitoring Rat1 in its application to telomere processing and in its application to DNA repair. Further, methods of controlling and monitoring Rat1 in its application to oncogenesis are provided. The disclosure provides methods of controlling and monitoring Rat1 in its application to genetically inherited syndromes of telomere shortening and in its application to aging. Methods of controlling and monitoring Rat1 in its application to radiotherapy and chemotherapy, and treatment of telomere shortening syndromes are provided herein.

### DEFINITIONS

As used herein and in the appended claims, the singular forms “a,” “and,” and “the” include plural referents unless the context clearly dictates otherwise. Thus, for example, reference to “a protein” includes a plurality of such proteins and reference to “the polynucleotide” includes reference to one or more polynucleotides and equivalents thereof, and so forth.

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

All publications mentioned herein are incorporated herein by reference in full for the purpose of describing and disclosing the methodologies, which are described in the publications, which might be used in connection with the description herein. The publications discussed above and throughout the text are provided solely for their disclosure prior to the filing date of the present application. Nothing herein is to be construed as an admission that the inventors are not entitled to antedate such disclosure by virtue of prior disclosure.

The term “polynucleotide” refers to a linear sequence of nucleotides. The nucleotides can be ribonucleotides, deoxyribonucleotides, or a mixture of both. Examples of polynucleotides contemplated herein include single- and double-stranded DNA, single- and double-stranded RNA (including miRNA), and hybrid molecules having mixtures of single- and double-stranded DNA and RNA.

The words “protein”, “peptide”, and “polypeptide” are used interchangeably to denote an amino acid polymer or a set of two or more interacting or bound amino acid polymers.

“Nucleic acid sequence” as used herein refers to an oligonucleotide, nucleotide or polynucleotide, and fragments and portions thereof, and to DNA or RNA of genomic or synthetic origin, which may be single- or double-stranded, and represent the sense or antisense strand. Similarly, “amino acid sequence” as used herein refers to peptide or protein sequence.

As used herein, a “nucleic acid probe” is a nucleic acid designed to hybridize to a target nucleic acid sequence. The nucleic acid probe is typically complementary to (e.g. perfectly complementary to) the target nucleic acid sequence. A “nucleic acid” means DNA, RNA, single-stranded, double-stranded, or more highly aggregated hybridization motifs, and any chemical modifications thereof, and includes ribonucleic acids. Modifications include, but are not limited to, those that provide other chemical groups that incorporate additional charge, polarizability, hydrogen bonding, electrostatic interaction, and functionality to the nucleic acid ligand bases or to the nucleic acid ligand as a whole. Such modifications include, but are not limited to, peptide nucleic acids, phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases isocytidine and isoguanidine and the like. Modifications can also include 3′ and 5′ modifications such as capping. The teun “RNA” means ribonucleic acid. The term “nucleotide” means a subunit of a DNA or RNA including the nitrogenous base, one or more phosphates, and ribose or deoxyribose, and includes those nucleotides forming part of a DNA or RNA strand wherein the phosphate forms part of a covalent linkage with an adjacent nucleotide (e.g. phosphodiester linkage). A “ribonucleotide” is nucleotide with a ribose ring.

Use of the term “complementary” in relation to the binding of nucleic acids is meant that a nucleic acid can form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, antisense or triple helix inhibition. Determination of binding free energies for nucleic acid molecules is well-known in the art (see, e.g., Turner et al., CSH Symp. Quant. Biol. LII:123-133 (1987); Frier et al., Proc. Nat. Acad. Sci. 83:9373-9377 (1986); Turner et al., J. Am. Chem. Soc. 109:3783-3785 (1987). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

A “deletion” is defined as a change in either nucleotide or amino acid sequence in which one or more nucleotides or amino acid residues, respectively, are absent.

An “insertion” or “addition” as used herein, is a change in a nucleotide or amino acid sequence which has resulted in the addition of one or more nucleotides or amino acid residues, respectively, as compared to naturally occurring sequences.

A “substitution” results from the replacement of one or more nucleotides or amino acids by different nucleotides or amino acids, respectively.

A “variant” in regard to amino acid sequences is used herein to indicate an amino acid sequence that differs by one or more amino acids from another, usually related amino acid. The variant may have “conservative” changes, wherein a substituted amino acid has similar structural or chemical properties (e.g. replacement of leucine with isoleucine). A variant may have “non-conservative” changes (e.g., replacement of a glycine with a tryptophan) or a variant may involve a change in charge (e.g. replacement of aspartic acid with arginine). Similar minor variations may also include amino acid deletions or insertions (i.e. additions), or both.

A “locus” as used herein is a fixed position on a chromosome that may be occupied by one or more genes. The locus of a gene on a chromosome is determined by its linear order relative to the other genes on that chromosome. A variant of the DNA sequence at a given locus is called “allele”.

Double-strand breaks (DSBs) and/or DNA damage, as used herein, refers to the case in which both strands of the double helix of DNA are severed at the same locus. In some embodiments, such breaks may be the result of an experimentally induced DSB at a defined site in the yeast or mammalian genome. In other embodiments, the DBBS are caused through treatment of cells with DNA damaging agents. The DSBs may be spontaneously occurring breaks in the genome, either in response to DNA replication errors or other defects in DNA metabolism.

“Telomere”, as used herein, are the ends of linear eukaryotic chromosomes with a special functional complex consisting of tandem repeated DNA sequences of duplex G (guanine)-rich repeats (with the G-rich strand extending beyond its complement to form an 3′ single-stranded overhang) and its associated proteins. In humans the telomeric repeat is 5″-TTAGGG-3′. Telomeres distinguish natural chromosome ends from DNA double-stranded breaks, and thus promote genome stability.

“Telomeric stability,” as used herein, refers to the tendency of a cell to form chromosomal aberrations and/or defects in cell cycle progression due to defects in normal telomere function, including problems in processing telomeres in the cell. Chromosomal aberrations due to abnormal telomere processing include, but are not limited to, telomere shortening or elongation, increased or decreased resection of the C-strand, loss of the 3′ G-rich overhang, an increased frequency in chromosome end-to-end associations, Lig4-dependent lethality, aneuploidy and/or polyploidy. Methods of assessing telomere stability are well known in the art. See, for example, Lendvay et al. (1996) Genetics 144, 1399-1412; Bertuch, A. A. and Lundblad, V. (2003) Mol. Cell. Biol., 23, 8202-8215; Nugent, et al. (1998). Curr Biol 8, 657-660; Nugent, C. I., Hughes, T. R., Lue, N. F., and Lundblad, V. (1996) Science 274, 249-252; Addinall et al. (2008) Genetics 180(4):2251-66; Myung et al., Mol. Cell. Biol., 24(11): 5050-5059 (2004); Hsu et al., Genes Dev., 14(22): 2807-28012 (200); and the assays provided in the Examples section below.

The term is “Rat1”, “terminase” or “terminase protein” as used herein refer to a polypeptide with the sequence of SEQ ID NO:1 or any homologues or alleles thereof. Examples for homologues include, but are not limited to, polypeptides encoded by the XRN2 gene and synthetically produced proteins that are identical in function and structure to the gene product of either the RAT1 or XRN2 gene.

“Terminase activity” as used herein, refers to the activity of a terminase protein and is defined as an exonuclease activity proceeding in a 5′ to 3′ direction. Without further limitation, the term “terminase activity” includes the catalytic exonuclease activity of an enzyme that functions to hydrolyze the phosphodiester bonds of a single-stranded deoxyribonuclease (DNA), with exonuclease activity proceeding in a 5′ to 3′ direction, and a catalytic exonuclease activity of an enzyme that functions to hydrolyze the phosphodiester bonds of one strand of a double-stranded deoxyribonuclease (DNA), with exonuclease activity proceeding in a 5′ to 3′ direction. The exonuclease activity may act on two types of DNA ends in cells: newly replicated chromosome termini, which are generated following replication of the genome during S phase, and double-strand breaks that occur throughout the genome as the result of DNA damage.

A “control terminase protein” is a terminase protein having the activity of an unaltered wild-type terminase protein. The activity of a control terminase protein may be used as a reference when determining the activity of an impaired terminase protein.

“Single-stranded exodeoxyribonuclease activity,” as used herein, refers to a catalytic exonuclease activity of an enzyme that functions to hydrolyze the phosphodiester bonds of a single-stranded deoxyribonuclease (DNA), with exonuclease activity proceeding in a 5′ to 3′ direction.

“Double-stranded exodeoxyribonuclease activity,” as used herein, refers to a catalytic exonuclease activity of an enzyme that functions to hydrolyze the phosphodiester bonds of one strand of a double-stranded deoxyribonuclease (DNA), with exonuclease activity proceeding in a 5′ to 3′ direction.

A “viral vector” is a viral-derived nucleic acid that is capable of transporting another nucleic acid into a cell. A viral vector is capable of directing expression of a protein or proteins encoded by one or more genes carried by the vector when it is present in the appropriate environment. Examples for viral vectors include, but are not limited to retroviral, adenoviral, lentiviral and adeno-associated viral vectors.

The term “recombinant” when used with reference, e.g., to a cell, or nucleic acid, protein, or vector, indicates that the cell, nucleic acid, protein or vector, has been modified by the introduction of heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under-expressed or not expressed at all. For further example, recombinant nucleic acid encode one or more genes, or portions thereof, that are not found within the native genes. For further example, recombinant proteins are proteins expressed by recombinant genes.

An “expression vector” is a nucleic acid construct, generated recombinantly or synthetically, with a series of specified nucleic acid elements that pelInit transcription of a particular nucleic acid in a host cell. The expression vector can be part of a plasmid, virus, or nucleic acid fragment. Typically, the expression vector includes a nucleic acid to be transcribed operably linked to a promoter.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, preferably 65%, 70%, 75%, 80%, 85%, 90%, 95%, 96%, 97%, 98%, 99% or even higher identity over a specified region), when compared and aligned for maximum correspondence over a comparison window, or designated region as measured using a sequence comparison algorithm or by manual alignment and visual inspection. This definition also refers to the complement of a nucleic acid sequence, as customary in the art.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters can be used, as known in the art.

A “comparison window,” as used herein, includes reference to a segment of any one of the number of contiguous nucleotide or amino acid positions in which a test sequence may be compared to a reference sequence of the same number of contiguous positions. In some embodiments, the two sequences are optimally aligned prior to calculation of sequence identity, as known in the art. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., CURRENT PROTOCOLS IN MOLECULAR BIOLOGY (Ausubel et al., eds., John Wiley & Sons, 1995 supplement)).

Exemplary algorithms suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein or with parameters known to the skilled artisan, to determine percent sequence identity for the nucleic acids and proteins of the invention. Software for performing BLAST analyses is publicly available, e.g., through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The term “transfection” or “transfecting” is defined as a process of introducing nucleic acid molecules into a cell by non-viral and viral-based methods. For non-viral methods of transfection any appropriate transfection method that does not use viral DNA or viral particles as a delivery system to introduce the nucleic acid molecule into the cell is useful in the methods described herein. Exemplary transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral based methods of transfection any useful viral vector may be used in the methods described herein. Examples for viral vectors include, but are not limited to retroviral, adenoviral, lentiviral and adeno-associated viral vectors.

The term “episomal” refers to the extra-chromosomal state of a plasmid in a cell. Episomal plasmids are nucleic acid molecules that are not part of the chromosomal DNA and replicate independently thereof.

A “test compound,” as used herein includes without limitation small molecules as well as biomolecules such as proteins (e.g. antibodies), peptides, nucleic acids, hormones and carbohydrates to be tested in the methods presented herein. A test compound may also encompass any type of formation of addition compounds and solvates and of substitutions which can be performed on the backbone chain of a substance, for example on an aromatic ring structure which is present in this chain. Substitutions of hydrogen atoms, halogen atoms, hydroxyl groups, amine groups, carboxylic acid groups or alkyl groups, or substitutions by such groups or atoms, can, for example, be performed. Biologically active variants of the compounds described herein for practicing the disclosure are particularly also encompassed by the methods of the disclosure. As used herein, an “analogue” refers to a compound in which one or more individual atoms or functional groups have been replaced, either with a different atom or a different functional, generally giving rise to a compound with similar properties. Indeed, a single compound, such as those described herein, may give rise to an entire family of analogues having similar activity and, therefore, usefulness according to the disclosure. Likewise, a single compound, such as those described herein, may represent a single family member of a greater class of compounds useful according to the disclosure. Accordingly, the disclosure fully encompasses not only the compounds described herein, but also analogues of such compounds, particularly those identifiable by methods commonly known in the art and recognizable to the skilled artisan. A “derivative”, as used herein, comprises a compound that is formed from a similar, beginning compound by attaching another molecule or atom to the beginning compound. Further, derivatives, according to the disclosure, encompass one or more compounds formed from a precursor compound through addition of one or more atoms or molecules or through combining two or more precursor compounds.

As used herein, the term “cancer” refers to all types of cancer, neoplasm or malignant tumors found in mammals, including leukemia, carcinomas and sarcomas. Exemplary cancers include cancer of the brain, breast, cervix, colon, head & neck, liver, kidney, lung, non-small cell lung, melanoma, mesothelioma, ovary, sarcoma, stomach, uterus and Medulloblastoma. Additional examples include, Hodgkin's Disease, Non-Hodgkin's Lymphoma, multiple myeloma, neuroblastoma, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine and exocrine pancreas, and prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). The P₃₈₈ leukemia model is widely accepted as being predictive of in vivo anti-leukemic activity. It is believed that a compound that tests positive in the P₃₈₈ assay will generally exhibit some level of anti-leukemic activity in vivo regardless of the type of leukemia being treated. Accordingly, the present invention includes a method of treating leukemia, and, preferably, a method of treating acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which is made up of a substance like the embryonic connective tissue and is generally composed of closely packed cells embedded in a fibrillar or homogeneous substance. Sarcomas which can be treated with a combination of antineoplastic mitochondrial oxidant and an anticancer agent include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas which can be treated with a combination of antineoplastic mitochondrial oxidant and an anticancer agent include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Exemplary carcinomas which can be treated with a combination of antineoplastic mitochondrial oxidant and an anticancer agent include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiernioid carcinoma, carcinoma epitheliale adenoides, exophytic carcinoma, carcinoma ex ulcere, carcinoma fibrosum, gelatiniformi carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

Cancer model organism, as used herein, is an organism exhibiting a phenotype indicative of cancer, or the activity of cancer causing elements, within the organism. The term cancer is defined above. A wide variety of organisms may serve as cancer model organisms, and include for example, cancer cells and mammalian organisms such as rodents (e.g. mouse or rat) and primates (such as humans).

### Compositions

In one aspect, a cell including a nucleic acid that encodes a terminase protein with (e.g., including) an impaired terminase activity is provided. The cell including the nucleic acid encoding the terminase protein including the impaired terminase activity may be a eukaryote. The term “eukaryote” refers to organisms that are distinguishable from “prokaryotes.” The term includes all organisms with cells that exhibit the usual characteristics of eukaryotes such as the presence of a true nucleus bounded by a nuclear membrane, within which lie the chromosomes, the presence of membrane-bound organelles, and other characteristics commonly observed in eukaryotic organisms. Thus the term includes, but is not limited to, such organisms as fungi, protozoa, and animals. In some embodiments, the cell is isolated (e.g. from an organism or an organ). In some embodiments, the cell is a yeast cell. In other embodiments, the cell is a mammalian cell. In other embodiments, the cell is a human cell. By “encoding” a terminase protein the cell may include a polynucleotide encoding a terminase protein or any variations thereof. The polynucleotide encoding the terminase protein may be part of the cell's genome or it may be present in an episomal state. The polynucleotide encoding the terminase protein or any variations thereof may be expressed transiently or may be stably expressed by the cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

An “impaired terminase activity” is defined as an impaired activity (e.g., decreased activity relative to the activity of a control) of a terminase protein at newly replicated chromosome termini and/or double-strand breaks. The impaired terminase activity deviates from the activity of a control terminase protein at newly replicated chromosome termini and/or double-strand breaks. As used herein, a control terminase protein includes, but is not limited to, a protein having the sequence of SEQ ID NO:1. In some embodiments, the terminase protein including an impaired terminase activity is a protein including alterations in SEQ ID NO:1. The alterations include any alterations in the sequence of the polynucleotide encoding the terminase protein. Examples for alterations in the sequence include, but are not limited to deletions, insertions and point mutations. Such sequence alterations can be obtained by methods well known in the art and include site-directed mutagenesis or linker-scanning mutagenesis. In some embodiments, the terminase protein having an impaired terminase activity is a protein fragment of a terminase protein. The terminase protein with an impaired terminase activity may include other naturally occurring terminase species and non-naturally occurring variants with impaired terminase activity. The terminase protein with an impaired terminase activity may also be obtained by de novo polypeptide synthesis.

In some embodiments, there is provided a terminase protein having impaired terminase activity, wherein the terminase protein has a defined level of sequence identity with respect to the sequence set forth for either of SEQ ID NOs:1-2. In some embodiments, the defined level of sequence identity is calculated with respect to SEQ ID NO:1. In some embodiments, the defined level of sequence identity is calculated with respect to SEQ ID NO:2. In some embodiments, the defined level of sequence identity is 70%, 75%, 80%, 85%, 90%, 95%, 96%, 97%, 98%, 99%, 99.1%, 99.2%, 99.3%, 99.4%, 99.5%, 99.6%, 99.7%, 99.8% or even greater, as calculated by methods described herein and/or as known in the art. In some embodiments, the defined level of sequence identity is in the range 70%-99.9%, 80%-99.9%, 90%-99.9%, 70%-99%, 80%-99%, 90%-99%, 70%-95%, 80%-95% or 90%-95%. In some embodiments, the sequence identity is calculated over a region of 20, 30, 40, 50, 60, 70, 80, 90, 100, 120, 140, 160, 180, 200, 300, 400, 500, 600, 700, or even greater, contiguous residues. In some embodiments, the sequence identity is calculated over the entire length of the reference terminase protein (e.g., SEQ ID NO:1 or SEQ ID NO:2). In some embodiments, the terminase protein having impaired terminase activity has 1, 2, 3, 4, 5, 6, 7, 8, 9, 10 or even more substitutions relative to SEQ ID NO:1. In some embodiments, the terminase protein having impaired terminase activity has 1, 2, 3, 4 or 5 substitutions. In some embodiments, the terminase protein having impaired terminase activity has 1 substitution. In some embodiments, the terminase protein having impaired terminase activity has 1, 2, 3, 4, 5, 6, 7, 8, 9, 10 or even more substitutions relative to a contiguous range of SEQ ID NO:1. In some embodiments, the substituted residues are contained within a contiguous region of 20, 30, 40, 50, 60, 70, 80, 90, 100, 120, 140, 160, 180, 200, 300, 400, 500, 600, 700, or even greater, contiguous residues.

In some embodiments, there exists at least one alteration in the terminase protein having impaired terminase activity relative to the sequence of SEQ ID NO:1. In some embodiments, the alteration is a substitution. In some embodiments, the alteration is a deletion. In some embodiments, the alteration is an insertion. In some embodiments, there exists one alteration (e.g., substitution) in the terminase protein having impaired terminase activity relative to the sequence of SEQ ID NO:1.

In some embodiments, an alteration is a substitution in the terminase protein having impaired terminase activity at a residue corresponding to any of residues 63, 79, 191, 241, 245, 253, 370, 699 or 724 of SEQ ID NO:1. The teems “residue corresponding” and the like refer, in the customary sense, to residue numbering using the numbering system of the reference polypeptide (e.g., either of SEQ ID NOs:1-2). In some embodiments, an alteration is a substitution at any of the residues corresponding to residues 63, 253 or 724 of SEQ ID NO:1. In some embodiments, an alteration is a substitution at any of the residues corresponding to residues 79, 191, 241, 245, 370 or 699 of SEQ ID NO:1. In some embodiments, the alteration is a substitution of aspartic acid for lysine at position 724 of SEQ ID NO:1 (i.e., D724K). As customary in the art, the term “AA¹XXXAA²” refers to substitution of amino acid “AA¹” at position “XXX” by amino acid “AA².” In some embodiments, the substitution is H63E, E253K or D724K. In some embodiments, the substitution is H63E In some embodiments, the substitution is E253K.

In some embodiments, an alteration is a substitution at any of the residues corresponding to any of residues 64, 80, 191, 241, 245, 253, 382, 611 or 636 of SEQ ID NO:2. In some embodiments, an alteration is a substitution at any of the residues corresponding to any of residues 64, 253 or 636 of SEQ ID NO:2. In some embodiments, an alteration is a substitution at any of the residues corresponding to any of residues 80, 191, 241, 245, 382 or 611 of SEQ ID NO:2.

In some embodiments, a substitution in a terminase protein having impaired terminase activity is a conservative amino acid substitution. The terms “conservative amino acid substitution” and the like, as customarily used in the art, refer to amino acids having similar chemical properties (e.g., size, hydrophobicity, hydrophilicity, charge, and the like). Exemplary conservative amino acid substitutions include the following groups: alanine and glycine; aspartic acid and glutamic acid; asparagine and glutamine; arginine and lysine; isoleucine, leucine, methionine and valine; phenylalanine, tyrosine and tryptophan; serine and threonine; and cysteine and methionine. See e.g., Creighton, PROTEINS, W.H. Freeman, 1984. In some embodiments, a substitution in a terminase protein having impaired terminase activity is a non-conservative amino acid substitution. The terms “non-conservative amino acid substitution” and the like, as customarily used in the art, refer to amino acid substitutions which are not conservative, as defined herein (e.g., D724K described herein).

In some embodiments of the cell provided herein which includes a nucleic acid that encodes a terminase protein with an impaired terminase activity, the cell is a recombinant cell. In some embodiments, the terminase protein is a recombinant protein. In some embodiments, the cell further includes a recombinant nucleic acid.

In some embodiments, there is provided a terminase protein as described herein (e.g., in the previous paragraphs under “Compositions”) and a nucleic acid encoding the terminase protein. In some embodiments, the nucleic acid is a recombinant nucleic acid, and the terminase protein is a recombinant terminase protein.

It is understood that the embodiments of the terminase protein (e.g., a recombinant terminase protein) disclosed herein are equally applicable to cell lines having a nucleic acid (e.g., a recombinant nucleic acid) encoding a terminase protein having impaired terminase activity, as well as methods disclosed herein which employ such cell lines, terminase proteins and/or nucleic acids.

By comparing an impaired terminase activity to a terminase activity of a control terminase protein at newly replicated chromosome termini, one may determine whether the impaired terminase activity is increased, decreased or the same as the activity of the control terminase protein at telomeres and/or double-stand breaks. For example, where the amount of the impaired terminase activity is greater than the activity of the control terminase protein at telomeres and/or double-strand breaks, the impaired terminase activity is increased. Where the amount of the impaired terminase activity is smaller than the activity of the control terminase protein at telomeres and or double-strand breaks, the impaired terminase activity is decreased. In some embodiments, the impaired terminase activity is increased when compared to a terminase activity of a control terminase protein. In other embodiments, the impaired terminase activity is decreased when compared to a terminase activity of a control terminase protein.

### Methods

Generation of Cells Expressing Terminase Proteins with Impaired Activity

In another aspect, a method for preparing a cell expressing a terminase protein with an impaired terminase activity is provided. The method includes introducing to the cell a nucleic acid encoding the terminase protein with the impaired terminase activity. The cell is allowed to express the terminase protein with the impaired terminase activity. And the impaired terminase activity in the cell is compared to a terminase activity in a control cell. In some embodiments, a control cell is a wild type cell that does not include a terminase with impaired terminase activity.

The process of “introducing” to the cell a nucleic acid encoding the terminase protein with the impaired terminase activity may be performed using any appropriate method known in the art. The nucleic acid may be introduced to the cell by transfection. Transfection methods as defined herein include, but are not limited to non-viral and viral-based transfection. Nonviral methods include calcium chloride transformation, electroporation, calcium phosphate treatment, liposome-mediated transformation, injection and microinjection, ballistic methods, immunoliposomes, polycation-nucleic acid conjugates, and naked DNA. The nonviral methods include plasmids and episomal vectors, typically with an expression cassette for expressing a protein, or RNA, and human artificial chromosomes. For example, nonviral vectors useful for expression of terminase proteins polynucleotides and polypeptides in mammalian cells include pcDNA3.1 and pEBVHis A, B, & C (Life Technologies, Carlsbad, Calif.). The viral methods include, but are not limited to viral vectors, virosomes and artificial virions. Examples of useful viral vectors are vectors based on retroviruses, adenoviruses, adeno-associated viruses, herpes viruses, SV40, papilloma virus, HBP Epstein Barr virus, vaccinia virus and Semliki Forest virus (SFV).

Allowing the cell to express the terminase protein with the impaired terminase activity may include expansion of the cell. Expansion as used herein includes the production of progeny cells. Expansion may occur in the presence of suitable media and cellular growth factors. Cellular growth factors are agents, which cause cells to migrate, differentiate, transform or mature and divide. They are polypeptides, which can usually be isolated from various normal and malignant mammalian cell types. Some growth factors are produced by genetically engineered microorganisms such as bacteria (E. coli) and yeasts.

The impaired terminase activity in the cell is compared to a terminase activity in a control cell (e.g. a wild type cell). The terminase activity in a control cell is the activity of a control terminase protein at newly replicated chromosome termini. In some embodiments, the control terminase protein is a wild type terminase, and the terminase activity in a control cell is the amount of terminase activity resulting from a wild type terminase. In some embodiments, the impaired activity is increased when compared to the terminase activity of the control cell. In other embodiments, the impaired terminase activity is decreased when compared to the terminase activity of the control cell.

In Vitro Assays

In another aspect, a method of determining whether a test compound modulates terminase activity in vitro is provided. The method includes combining a test compound, a terminase protein, and a double-stranded deoxynucleotide substrate in a single reaction mixture in vitro under conditions conducive to terminase activity. Sufficient time is allowed for the terminase protein to react with the double-stranded deoxynucleotide substrate to form a hydrolyzed double-stranded deoxynucleotide product. An amount of the hydrolyzed double-stranded deoxynucleotide product is detected and the amount is compared to a control amount of hydrolyzed double-stranded deoxynucleotide product, thereby determining whether the test compound modulates terminase activity in vitro.

The single reaction mixture is typically provided in a single reaction vessel such as a well of a multi-well plate or a glass container. One skilled in the art will immediately recognize that the conditions under which the test compound is combined with the terminase protein and the single- or double-stranded deoxynucleotide substrate should be conditions in which the terminase protein single or double-stranded exodeoxyribonuclease activity is known to be active (i.e. conditions conducive to terminase protein single or double-stranded exodeoxyribonuclease activity). These conditions may be easily ascertained from the description provided herein. See e.g., Examples section below.

A double-stranded deoxynucleotide substrate is a double-stranded DNA having phosphodiester linkages and at least one double-stranded end having one 5′ end strand and one 3′ end strand. The double-stranded deoxynucleotide substrate may have any appropriate length. For example, in some embodiments, the double-stranded deoxynucleotide substrate is approximately 50 base pairs and is of synthetic origin. The double-stranded deoxynucleotide substrate may also be derived from a plasmid that has been linearized (e.g. at a restriction site).

A single-stranded deoxynucleotide substrate is a single-stranded DNA having phosphodiester linkages and a 5′ end a 3′ end. The single-stranded deoxynucleotide substrate may have any appropriate length. For example, in some embodiments, the single-stranded deoxynucleotide substrate is approximately 50 base pairs and is of synthetic origin. The single-stranded deoxynucleotide substrate may further be the 5′ protrusion of a double-stranded deoxynucleotide.

In some embodiments, the double-stranded deoxynucleotide substrate is a blunt ended double-stranded deoxynucleotide substrate. A blunt ended double-stranded deoxynucleotide substrate is a double-stranded deoxynucleotide substrate having at least one double-stranded end having one 5′ end strand and one 3′ end strand, wherein the last nucleotide in the 5′ end strand is hydrogen bonded (or paired) to the last nucleotide of the 3′ end strand (i.e. a blunt end) thereby leaving no nucleotide overhang. Where the double-stranded deoxynucleotide substrate is a blunt ended double-stranded deoxynucleotide substrate, the action of the terminase may result in a 3′-overhang double-stranded deoxynucleotide product. A 3′-overhang double-stranded deoxynucleotide product is a hydrolyzed product of a blunt ended double-stranded deoxynucleotide substrate in which the blunt end is converted to a double-stranded end having one 5′ end strand and one 3′ end strand, wherein the last nucleotide in the 3′ end strand is not hydrogen bonded (or paired) to the last nucleotide of the 5′ end strand and the last nucleotide of the 5′ end is hydrogen bonded (or paired) to a nucleotide on the complimentary strand. In some embodiments, the hydrolyzed double-stranded deoxynucleotide product is a 3′-overhang double-stranded deoxynucleotide product.

Sufficient time should be allowed for the terminase protein to react with the single- or double-stranded deoxynucleotide substrate to form a hydrolyzed single- or double-stranded deoxynucleotide product. Depending upon whether the test compound modulates terminase protein single- or double-stranded exodeoxyribonuclease activity, the single- or double-stranded deoxynucleotide substrate may or may not actually form a hydrolyzed single- or double-stranded deoxynucleotide product. Therefore, the amount of time sufficient for the terminase protein to react with the single- or double-stranded deoxynucleotide substrate to form a hydrolyzed single- or double-stranded deoxynucleotide product within the context of the present method is actually the amount of time sufficient for the terminase protein to react with the single- or double-stranded deoxynucleotide substrate in the absence of the test compound under the same reaction conditions (i.e. a control experiment). Thus, the amount of time sufficient for a terminase protein to react with the single- or double-stranded deoxynucleotide substrate in the absence of the test compound is the amount of time sufficient for the reaction to take place when performing the method of determining whether a test compound modulates terminase single- or double-stranded exodeoxyribonuclease activity in vitro. The amount of time sufficient for the terminase protein to react with the single- or double-stranded deoxynucleotide substrate can be easily determined by one skilled in the art given the guidance provided in the Examples section below and the general knowledge in the art of exonuclease assays. The amount of time sufficient will depend upon the specific reaction conditions chosen, such as the amount of terminase protein, the amount of single- or double-stranded deoxynucleotide substrate, and the physical characteristics of the single- or double-stranded deoxynucleotide substrate.

The amount of the hydrolyzed single- or double-stranded deoxynucleotide product is detected and compared to a control amount of hydrolyzed single- or double-stranded deoxynucleotide product. A control amount of hydrolyzed single- or double-stranded deoxynucleotide product may be easily obtained using a control experiment. For example, in some embodiments a control experiment includes combining a terminase protein with a single- or double-stranded deoxynucleotide substrate in the absence of a test compound under the conditions to be used in the method of determining whether a test compound modulates terminase single- or double-stranded exodeoxyribonuclease activity in vitro. After a sufficient time is allowed for the terminase protein to react with the single- or double-stranded deoxynucleotide substrate, the amount of the hydrolyzed single- or double-stranded deoxynucleotide product in the absence of test compound, which is the control amount hydrolyzed single- or double-stranded deoxynucleotide product, is thereby detected.

By comparing the amount of the hydrolyzed single- or double-stranded deoxynucleotide product to the control amount of hydrolyzed single- or double-stranded deoxynucleotide product, one may determine whether the test compound modulates Rat1 exodeoxyribonuclease activity in vitro. For example, where the amount of the hydrolyzed single- or double-stranded deoxynucleotide product is greater than the control amount of hydrolyzed single- or double-stranded deoxynucleotide product, the test compound modulates the terminase protein single- or double-stranded exodeoxyribonuclease activity by agonizing the terminase protein single- or double-stranded exodeoxyribonuclease activity. Where the amount of the hydrolyzed single- or double-stranded deoxynucleotide product is less than the control amount of hydrolyzed single- or double-stranded deoxynucleotide product, the test compound modulates the terminase protein single- or double-stranded exodeoxyribonuclease activity by antagonizing (e.g. inhibiting) the terminase protein single- or double-stranded exodeoxyribonuclease activity. And where the amount of the hydrolyzed single- or double-stranded deoxynucleotide product is approximately the same as the control amount of hydrolyzed single- or double-stranded deoxynucleotide product, the test compound does not modulate the terminase protein single- or double-stranded exodeoxyribonuclease activity.

Methods of detecting of the amount of hydrolyzed single- or double-stranded deoxynucleotide products are well known in the art. For example, a detectable nucleic acid probe may be designed that hybridizes to the hydrolyzed double-stranded deoxynucleotide products (e.g. one of the strands of the hydrolyzed double-stranded deoxynucleotide product) but does not hybridize to the double-stranded deoxynucleotide substrate. Once the detectable nucleic acid probe is bound to the hydrolyzed double-stranded deoxynucleotide product, the double-stranded deoxynucleotide substrate may be optionally separated from the reaction mix and the amount of hydrolyzed double-stranded deoxynucleotide product is detected (e.g. quantitation by quantitative PCR techniques or using gel hybridization). A specific example of a detection assay for a 3′-overhang double-stranded deoxynucleotide product is set forth below in the Examples section. Briefly, in gel hybridization of a ³²P-labeled nucleic acid probe to the 3′-overhang double-stranded deoxynucleotide product is performed and detected using film or phosphoimager.

The amount of hydrolyzed single- or double-stranded deoxynucleotide products detected will depend upon the degree and type of modulation, if any, the test compound exhibits when tested with the terminase protein. For example, in some embodiments, there is no detectable amount of hydrolyzed double-stranded deoxynucleotide product, thereby indicating strong antagonistic action of the test compound against terminase. In other embodiments, there is no detectable amount of hydrolyzed single-stranded deoxynucleotide product, thereby indicating strong antagonistic action of the test compound against terminase.

As indicated above, the methods provided herein for determining whether a test compound modulates terminase single- or double-stranded exodeoxyribonuclease activity in vitro include methods for determining whether a test compound agonizes (e.g. increases) terminase single- or double-stranded exodeoxyribonuclease activity vitro and methods for determining whether a test compound antagonizes (e.g. decreases or inhibits) terminase single- or double-stranded exodeoxyribonuclease activity vitro.

In Vivo Assays

In another aspect, a method is provided for determining whether a test compound (e.g. an in vitro terminase modulating test compound) modulates terminase double-stranded exodeoxyribonuclease activity in a cell. In some embodiments, the method includes contacting a test compound (e.g. an in vitro terminase modulating test compound) with a cell. The amount of telomeric stability in the cell is determined and compared to a control amount of telomeric stability thereby determining whether the test compound (e.g. the in vitro terminase modulating test compound) modulates terminase double-stranded exodeoxyribonuclease activity in the cell. Where the amount of telomeric stability is assessed, the method may be referred to as a method of determining whether a test compound (e.g. an in vitro terminase modulating test compound) modulates telomeric stability in a cell.

An “in vitro terminase modulating test compound,” as used herein, refers to a test compound that is known (e.g. has been shown or previously demonstrated) to modulate terminase protein double-stranded exodeoxyribonuclease activity in vitro according to the methods set forth in the previous section. The method includes combining a test compound, a terminase protein, and a double-stranded deoxynucleotide substrate in a single reaction mixture in vitro under conditions conducive to terminase protein double-stranded exodeoxyribonuclease activity. Sufficient time is allowed for the terminase protein to react with the double-stranded deoxynucleotide substrate to form a hydrolyzed double-stranded deoxynucleotide product. The amount of the hydrolyzed double-stranded deoxynucleotide product is detected and compared to a control amount of hydrolyzed double-stranded deoxynucleotide product, thereby determining that the test compound is an in vitro terminase modulating test compound (also referred to herein as an in vitro terminase double-stranded exodeoxyribonuclease activity modulating test compound). In vitro methods are described in detail above. The characteristics of the in vitro methods described above are equally applicable to the identification of an in vitro terminase modulating test compound.

Any appropriate cell may be used to determine whether a test compound modulates terminase double-stranded exodeoxyribonuclease activity in a cell. In some embodiments, the cell is a eukaryotic cell. The eukaryotic cell may be mammalian cell (e.g. a human cell). The cell may form part of an organ or organism. In some embodiments, the cell does not form part of an organ or organism.

The amount of telomeric stability in the cell may be determined using any appropriate method. Telomeric stability, as used herein, refers to the tendency of a cell to form chromosomal aberrations upon division due to problems in processing telomeres in the cell. Chromosomal aberrations due to abnormal telomere procession includes, for example, increased sister telomere loss (STL), telomere shortening, increased isolated telomeric DNA, loss of telomeres replicated by lagging strand DNA synthesis, chromosome end to end association, centromere formation, a decrease in the number or sizes of G-tail ends, aneuploidy, and/or polyploidy. Methods of assessing telomere stability are well known in the art. See, for example, Agata et al., Current Biology, 12(19):1635 (2002); Papadopoulas et la., Int. J. of Cancer, 99(2): 193-200 (2002); Myung et al., Mol. Cell. Biol., 24(11): 5050-5059 (2004); Gilley et al., Proc. Nat. Acad. Sci., 98(26): 15084 (2001); d'Adda di Fagagna et al., Current Biology, 11(15): 1192-1196 (2001); Hsu et al., Genes Dev., 14(22): 2807-28012 (200); Ohyashiki et al., Clin. Cancer Res., 5: 1155-1160 (1999); Pandita et al., Mol. and Cell. Biol., 26(5): 1850-1864 (2006); and the G-tail assays provided in the Examples section below.

The amount of telomeric stability in the cell is determined and compared to a control amount of telomeric stability. A control amount of telomeric stability product may be easily obtained using a cellular control experiment. For example, a cellular control experiment includes determining the amount of telomeric stability in a cell in the absence of the test compound (e.g. the in vitro terminase modulating test compound) using the same method to determine the amount of telomeric stability, the same cell type, and the same conditions to be used in the method of determining whether a test compound (e.g. an in vitro terminase modulating test compound) modulates terminase double-stranded exodeoxyribonuclease activity in a cell. The amount of telomeric stability in the absence of test compound, which is the control amount of telomeric stability, is thereby detected.

By comparing the amount of telomeric stability to the control amount of telomeric stability, one may determine whether the test compound modulates terminase double-stranded exodeoxyribonuclease activity in a cell. For example, where the amount of telomeric stability is greater than the control amount of telomeric stability, the test compound modulates terminase double-stranded exodeoxyribonuclease activity by agonizing the terminase double-stranded exodeoxyribonuclease activity in a cell. Where the amount of telomeric stability is less than the control amount of telomeric stability, the test compound modulates the terminase protein double-stranded exodeoxyribonuclease activity by antagonizing (e.g. inhibiting) the terminase protein double-stranded exodeoxyribonuclease activity in the cell. And where the amount of the telomeric stability is approximately the same as the control amount of telomeric stability, the test compound does not modulate the terminase protein double-stranded exodeoxyribonuclease activity in the cell.

As indicated above, the methods provided herein for determining whether a test compound (e.g. an in vitro terminase modulating test compound) modulates terminase double-stranded exodeoxyribonuclease activity in a cell include methods for determining whether a test compound agonizes (e.g. increases) terminase double-stranded exodeoxyribonuclease activity in a cell and methods for determining whether a test compound antagonizes (e.g. decreases or inhibits) terminase double-stranded exodeoxyribonuclease activity in a cell. Likewise, methods of determining whether a test compound (e.g. an in vitro terminase modulating test compound) modulates telomeric stability in a cell include methods of determining whether a test compound (e.g. an in vitro terminase modulating test compound) agonizes (e.g. increases) telomeric stability in a cell and methods of determining whether a test compound (e.g. an in vitro terminase modulating test compound) antagonizes (e.g. decreases or inhibits) telomeric stability in a cell.

In one aspect, a method is provided to determine whether a test compound modulates terminase activity in a cell. The method includes contacting the test compound with a cell. An amount of telomeric stability in the cell is determined and the amount is compared to a control amount of telomeric stability, thereby determining whether the test compound modulates terminase activity in the cell.

Methods Related to Cancer

In another aspect, a method of determining whether a test compound is a terminase modulating anticancer compound is provided. The method includes determining whether a test compound modulates terminase activity in vitro according to the method provided herein. In some embodiments, the test compound may already be known or suspected to modulate terminase activity. The test compound is administered to a cancer model organism. It is determined whether the test compound exhibits anticancer properties in the cancer model organism.

In some embodiments, the method further includes, after determining that the test compound modulates terminase exodeoxyribonuclease activity in vitro (i.e. that the test compound is an in vitro terminase modulating test compound), the test compound is evaluated to determine whether the test compound (e.g. an in vitro terminase modulating test compound) modulates terminase double-stranded exodeoxyribonuclease activity in a cell, as set forth above in the In Vivo Assays section. For example, the test compound is contacted with a cell. The amount of telomeric stability in the cell is compared to a control amount of telomeric stability, thereby determining whether the test compound modulates terminase double-stranded exodeoxyribonuclease activity in said cell. The determination of whether the test compound modulates terminase activity double-stranded exodeoxyribonuclease activity in a cell is typically performed before administering the test compound to a cancer model organism.

A “cancer model organism,” as used herein, is an organism exhibiting a phenotype indicative of cancer, or the activity of cancer causing elements, within the organism. The term cancer is defined above in the Definitions section. A wide variety of organism may serve as cancer model organisms, and include for example, mammalian organisms such as rodents (e.g. mouse or rat) and primates (such as humans). Thus, in some embodiments, the method of determining whether a test compound is a terminase modulating anticancer compound is a clinical trial for a terminase modulating anticancer compound.

In another aspect, a method for determining whether a test compound is a terminase modulating anticancer compound is provided. The method includes administering the test compound to a cancer model organism and determining whether the test compound exhibits anticancer properties in the cancer model organism.

Methods Related to Double-Strand Break Repair

In another aspect, a method is provided for determining whether a test compound modulates DNA repair of a double-strand break of a double-stranded nucleic acid in a cell by modulating terminase activity. The method includes determining whether the test compound (e.g. an in vitro terminase modulating test compound as described above) modulates terminase activity in vitro according to the methods described in the In Vitro Assays section above. The method further includes contacting the test compound (e.g. an in vitro terminase modulating test compound) with a cell and determining an amount of DNA repair in the cell. The amount of DNA repair in the cell is compared to a control amount of DNA repair in the cell thereby determining whether the test compound increases DNA repair of a double-strand break of a double-stranded nucleic acid in the cell by increasing terminase activity.

Any appropriate cell may be used to determine whether a test compound modulates DNA repair of a double-strand break of a double-stranded nucleic acid in a cell by modulating terminase activity. In some embodiments, the cell is a eukaryotic cell. The eukaryotic cell may be mammalian cell (e.g. a human cell). The cell may form part of an organ or organism. In some embodiments, the cell does not form part of an organ or organism.

The amount of DNA repair in the cell may be assayed using known techniques in the art, such as those set forth in Hu et al., Cancer Research, 65:8613-8616 (2005); Pierce et al., Genes Dev, 13:2633-8 (1999); Taghian et al., Mol Cell Biol, 17:6386-93 (1997); and Slebos et al., Biochem Biophys Res Commun, 281(1): 212-9 (2001).

The amount of DNA repair in the cell is determined and compared to a control amount of DNA repair. A control amount of DNA repair product may be easily obtained using a cellular control experiment. For example, a cellular control experiment includes determining the amount of DNA repair in a cell in the absence of the test compound using the same method to determine the amount of DNA repair, the same cell type, and the same conditions to be used in the method of determining whether a test compound modulates DNA repair of a double-strand break of a double-stranded nucleic acid in a cell by modulating terminase activity. The amount of DNA repair in the absence of test compound, which is the control amount of DNA repair, is thereby detected.

By comparing the amount of DNA repair to the control amount of DNA repair, one may determine whether a test compound modulates DNA repair of a double-strand break of a double-stranded nucleic acid in a cell by modulating terminase activity. For example, where the amount of DNA repair is greater than the control amount of DNA repair, the test compound agonizes (e.g. increases) DNA repair of a double-strand break of a double-stranded nucleic acid in a cell by agonizing (e.g. increasing) terminase activity. Where the amount of DNA repair is less than the control amount of DNA repair, the test compound antagonizes (e.g. decreases or inhibits) DNA repair of a double-strand break of a double-stranded nucleic acid in a cell by antagonizing (e.g. decreases or inhibits) terminase activity. And where the amount of DNA repair is the same as than the control amount of telomeric stability, the test compound does not modulate DNA.

The methods provided herein for determining whether a test compound (e.g. an in vitro terminase modulating test compound) modulates DNA repair of a double-strand break of a double-stranded nucleic acid in a cell by modulating terminase activity include methods for determining whether a test compound agonizes (e.g. increases) DNA repair in a cell and methods for determining whether a test compound antagonizes (e.g. decreases or inhibits) DNA repair in a cell.

In another aspect, a method is provided to determine whether a test compound modulates double-strand break-specific terminase activity in a cell. The method includes contacting the test compound with a cell. An amount of DNA repair in the cell is determined and the amount is compared to a control amount of DNA repair in the cell. Thereby it is determined whether the test compound modulates DNA repair of a double-strand break of a double-stranded nucleic acid in the cell by modulating double-strand break-specific terminase activity.

### Kits

In one aspect, a kit for determining whether a test compound modulates terminase activity in vitro is provided. The kit includes a terminase protein and a double-stranded deoxynucleotide substrate. In some embodiments, the kit further includes additional features disclosed above in the In Vitro Assays section.

### EXAMPLES

### Example 1

### Partial Tandem Affinity Purification (TAP) of the S. cerevisiae Rat1 Protein

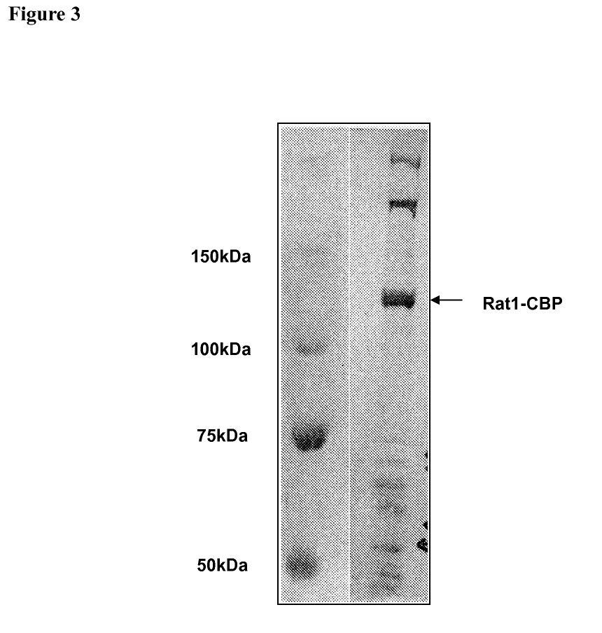

In this example, Rat1 was purified from S. cerevisiae yeast cultures using a modified version of the tandem affinity purification (TAP) technique (Rigaut, G., et al., Nat Biotechnol, 17(10):1030-2 (1999)). An S. cerevisiae strain expressing RAT1-TAP (met15-Δ his 3-Δ1 ura3-Δ leu2-Δ RAT1::TAP-HIS3MX6), constructed as described in Ghaemmaghami, S et al., Nature 425:737-741 (2003), was purchased from Open Biosystems (Huntsville, Ala.). 2 L YPD cultures were grown to OD₆₀₀=1.0, pelleted and washed 2× with dH₂O, 1× with TAP lysis buffer, 4° C. Cell pellets were resuspended in one volume cold TAP lysis buffer (50 mM Tris pH8, 150 mM KCl, 10% glycerol, protease inhibitors) and ground in a mortar under liquid-N₂ to disrupt cell walls. Lysates were thawed and cleared at 19000 rpm, 30 min, 4° C. Cleared lysates were adjusted to 0.1% NP40 and 5 mM EDTA. TAP proteins were precipitated through incubation with 200 μl IgG Sepharose™, 2 hr, 4° C. After washing the beads three times with 10 ml cold IPP 150, and once with TEV buffer, the precipitated protein was cleaved from the IgG Sepharose™ by incubation with 10 μl TEV protease in 1 ml TEV cleavage buffer at RT, 90 mM. The product of the TEV cleavage reaction was dialyzed against a storage buffer (20 mM Tris pH7.5, 200 mM NaCl, 60% glycerol, 0.1 mM EDTA), 4° C. overnight and subsequently stored at −20° C. The Rat1 —CBP protein was assessed using the CBP Detection Assay (Stratagene, Agilent Technolies, Santa Clara, Calif.) according to the manufacturer's protocol (FIG. 3), which detected the CBP (calmodulin binding peptide) portion of the TAP tag that remained after TEV cleavage.

### Example 2

### The S. cerevisiae Rat1 Protein Exhibits 5′→3′ DNA Exonuclease Activity with a Single-Stranded DNA Oligomer

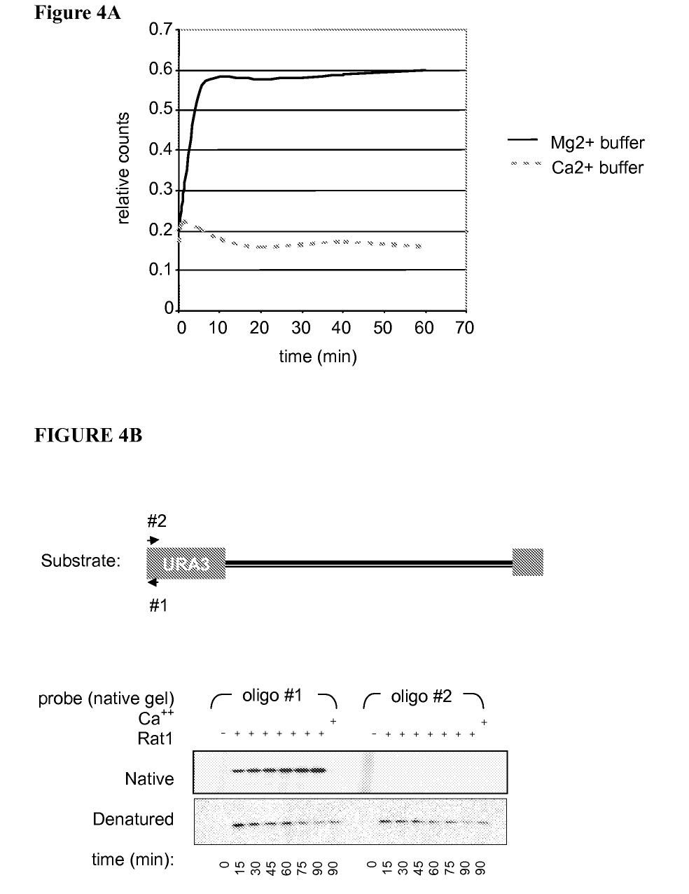

The Rat1 preparation described in Example 1 was tested for 5′→3′ exonuclease activity on a single-stranded DNA oligo substrate, using a protocol designed to assay activity of Sep1, a closely related paralog to Rat1 (Johnson, A. W. and R. D. Kolodner, J Biol Chem, 269(5):3664-72 (1994)). In this assay, the substrate oligo was labeled at the 5′ end with ³²P and the nuclease activity was measured as the release of TCA-soluble ³²P counts. A 30 μl reaction contained 1 μl 5′-end labeled oligo (GGG TGT GGG TGT GTG TGG TGG G), 1× Rat1 reaction buffer (33 mM Tris pH7.5, 13 mM MgCl₂, 1.8 mM DTT), and 2 μl Rat1 from the partial TAP purification described in the previous example. To determine if the activity was inhibited by Ca⁺⁺, a parallel set of reactions were perfoinied with Ca⁺⁺ buffer (33 mM Tris pH 7.5, 5 mM CaCl₂, 2 mM DTT, 100 μg/ml BSA) in place of Rat1 reaction buffer. Reactions were incubated at 30° C. for up to 60 minutes, with aliquots removed at various time points and stopped by the addition of 12.5 μl 100 mM EDTA and 42.5 μl 0.8 M TCA. Products were precipitated on ice for 10 min, and then pelleted at 12,000 rpm for 10 min. 60 μl of the supernatant, containing the TCA soluble products, were transferred to scintillation fluid and counted. The results of such an experiment are shown in FIG. 4A, which shows the release of TCA soluble ³²P counts over time, in the presence of Mg²⁺(solid line) or Ca⁺⁺ (dashed line). In the presence of Mg²⁺, the reaction went to completion in under 10 min; the reaction required the addition of Rat1 and was inhibited by Ca⁺⁺. This result illustrated that Rat1 is able to cleave nucleotide(s) from the 5′ terminus of a single-stranded DNA substrate.

### Example 3

### The S. cerevisiae Rat1 Protein Exhibits Strand-Specific 5′→3′ Exonuclease Activity with a Duplex DNA Substrate

The Rat1 preparation described in Example 1 was tested for 5′→3′ exonuclease activity on a duplex DNA substrate, using yet another assay developed for Sep1p (Zhang, Z., et al., J Biol Chem, 273(9):4950-6 (1998)). In this assay, Rat1 is incubated with a linearized plasmid (YEplac195, digested to completion with the restriction enzyme StuI, which cuts once in the plasmid), and resection is detected by resolving products that have been annealed with ³²P-labeled strand-specific oligomers on an agarose gel. Reactions were performed in either 1× Rat1 reaction buffer or Ca⁺⁺ buffer; a typical 100 ml reaction contained 1 μg Stu1-digested Yeplac195 and 10 μl Rat1 preparation. Reactions were incubated at 30°, with 10 μl aliquots removed every 15 mM for up to 90 min; reactions were stopped by the addition of 4 μl 100 mM EDTA on ice. The products were EtOH precipitated, resuspended and hybridized to 1 μl ³²P-labeled oligomers and run on a 0.7% TBE agarose gel (without EtBr), dried, and exposed to film or phosphoimager screen to record the native gel signal. The dried gel was then washed in denaturation solution (150 mM NaCl, 0.5 M NaOH) to remove probe, neutralized (150 mM NaCl, 0.5 M Tris pH8.0), and hybridized to ³²P-labeled oligo #2. The hybridized gel was washed in 0.25×SSC and exposed to film or phosphoimager screen to record denatured signal. The sequences of the oliogomers used are oligo #1 (5′-CCTCTAGGTTCCTTTGTTAC-3′) (SEQ ID NO:3) and oligo #2 (5′-GTAACAAAGGAACCTAGAGG-3′) (SEQ ID NO:4). This assay demonstrated that the Rat1 protein was capable of strand-specific DNA exonuclease activity on a blunt duplex DNA molecule, by degrading DNA specifically from one strand, in a 5′→3′ direction (FIG. 4B). Activity was dependent on addition of the Rat1 preparation and inhibited by the presence of Ca⁺⁺.

### Example 4

### Peptide Antibodies that Recognize the S. cerevisiae Rat1 Protein

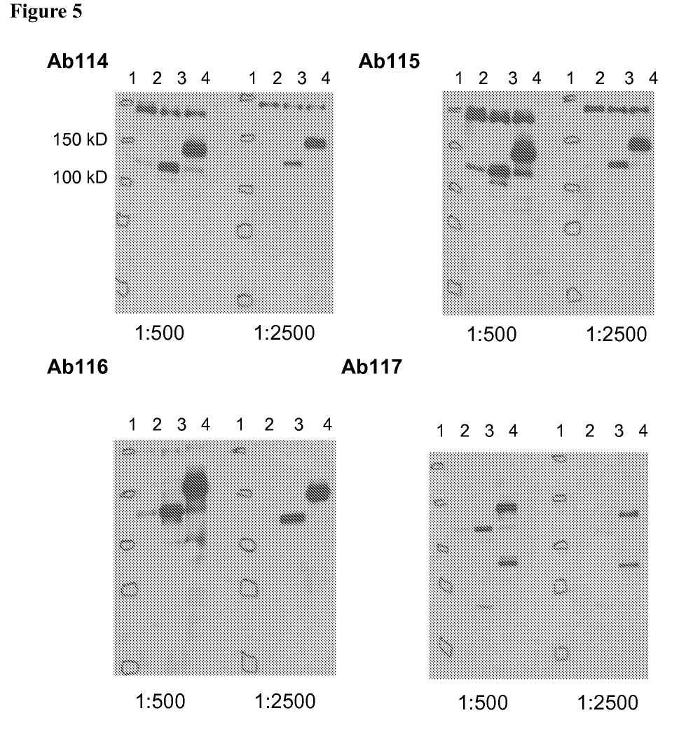

Custom anti-peptide antibodies were generated by ProSci Incorporated, against two regions of the S. cerevisiae Rat1 protein: YSGNSYSRNNKQSRYDNSRANRR (Ab 114 and 115 (SEQ ID NO:5), corresponding to two individual rabbits) and NDRENSETTEVSRD (SEQ ID NO:6) (Ab 116 and 117, two additional rabbits). Antibody preparations were evaluated for their ability to detect the native Rat1 protein by western analysis. The levels of endogenous Rat1 protein present in a wild type yeast strain were compared with the levels of Rat1 protein in a strain carrying a plasmid that expressed the Rat1 protein at high levels, under the control of the constitutive ADH promoter (pVL3271), and also compared to a strain expressing a version of the Rat1 protein that was fused to the Gal4 activation domain, also under the control of the ADH promoter (pVL3270). Cell pellets from these three strains were lysed by grinding in a mortar under liquid-N₂ to disrupt cell walls. Lysates were thawed and cleared at 14,000 rpm for 15 min at 4° C. A panel of four identical 6% SDS-PAGE acrylamide gels were run, loaded with approximately 30 μg of total protein from each of the three extracts, along with size markers, and transferred overnight onto nitrocellulose. Membranes were incubated in 5% milk TBS-T for one hour, rinsed in TBS, incubated for one hour at room temperature in 5 ml PBS casein solution plus a anti-Rat1 peptide antibody at either 1:500 or 1:2500 dilution, rinsed in TBS-T and incubated for 30 min at room temperature in 5 ml PBS casein solution plus the secondary, anti-rabbit HRP, at 1:5000. Signal was visualized using ECL (Amersham), according to manufacturers directions, and exposures from 1 to 5 minutes were used to evaluate the ability of the four antibodies to detect the level of Rat1 protein (FIG. 5).

### Example 5

### Rat1 Localizes to Experimentally Generated Double-Strand Breaks

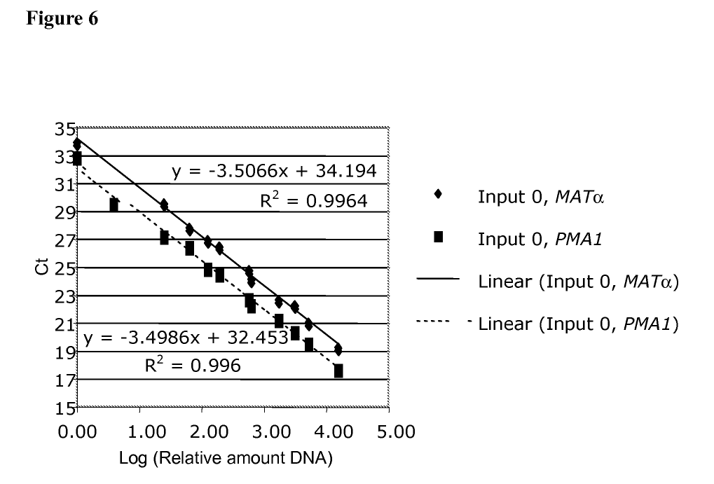

The in vivo response to a double-strand break can be experimentally assessed in a specialized yeast strain (JKM 179; MAT α hml-Δ::ADE1 hmr-Δ::ADE1 ade1-110 leu2, 3-112 lys5 trp1-Δ::hisG ura3-52 ade3::GAL10:HO; Valencia et al., Nature, 2001 414:666-669). In this strain, the HO enzyme is expressed under the control of the inducible GAL10 promoter: when the HO enzyme is induced upon addition of galactose to the medium, this results in the generation of a double-strand break at a single site in the yeast genome, the MAT α locus. This system allows monitoring resection of DNA at this experimentally induced double-strand break, as well as association of predicted proteins. To examine whether Rat1 localizes to DNA at a newly generated double-strand break (DSB), as would be expected of a nuclease acting in a DSB repair pathway, a chromosome immuno-precipitation assay (ChIP) was used to monitor association of the Rat1 protein at HO-generated double-strand breaks, using the anti-Rat1 peptide Ab116 antibody described in Example 4. ChIP samples were monitored by Real-time Q-PCR (quantitative PCR), for association with sequences in the MAT α locus, immediately adjacent to the HO-induced break, as well as at the PMA1 locus, which had been previously shown to associate with the Rat1 protein. The primers for the MAT α locus were: 5′-AGATGTGTTTGTACATTTGGCCTTATA-3′ (SEQ ID NO:7) and 5′-TACCCTTATCTACTTGCCTCTTTTGTT-3′ (SEQ ID NO:8), and the primer set for the PMA1 locus were: 5′-ATGAACCACAGTACATCATATTAAGACGTA-3′ (SEQ ID NO:9) and 5′-GAAGAAATTTCGATGCGATTAACC-3 (SEQ ID NO:10). The two primer sets were initially evaluated using the serial dilutions of input genomic DNA samples, in order to evaluate primer efficiency, by plotting the Ct value (“Threshold cycle”) against log(relative amounts of DNA). Evaluation of the data for two independent repeats demonstrated that the two primer sets work with high, and similar, efficiency (FIG. 6).

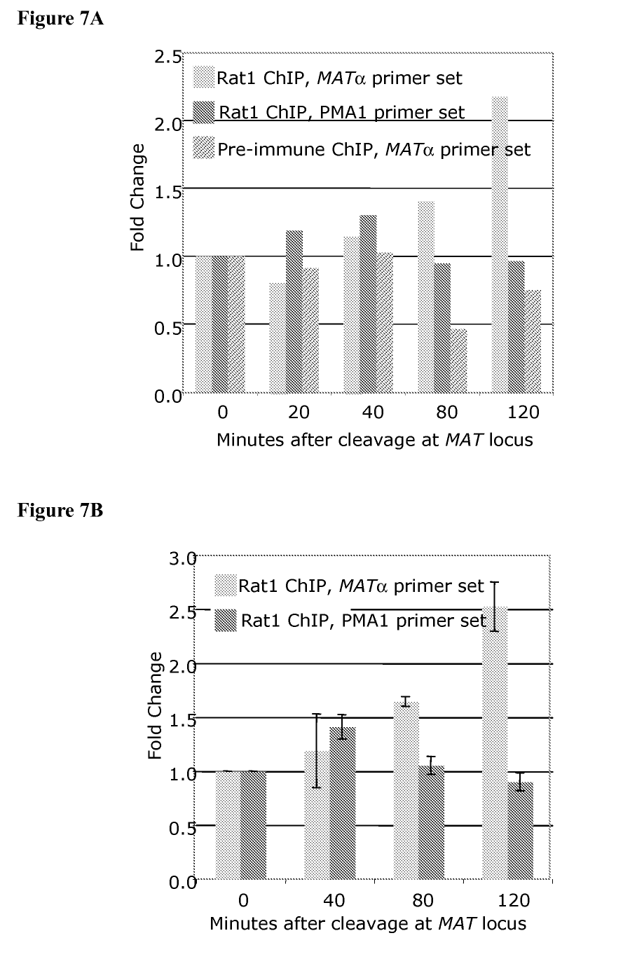

The ChIP protocol was adapted from Aparicio, et al CURRENT PROTOCOLS IN MOLECULAR BIOLOGY, F. M. Ausubel, et al., Eds. (John Wiley and Sons, Inc., New York, 1999), pp. 21.3.1-21.3.12. A 200 ml culture of the JKM179 strain was grown in YP-Lactate medium to OD=0.6, and 2% galactose was added to induced HO-mediated cleavage at MAT locus; 40 ml samples were removed just prior to the addition of galactose (t=0) and 20 (or 40) minute intervals for up to 2 hours. Cells were fixed by adding 1.1 ml 37% formaldehyde for 20 min at RT. Cross linking reaction was stopped by the addition of 6 ml 2.5 M glycine, RT for 5 min. After all samples were collected, fixed cells were pelleted and washed twice in 1× TBS, and once in FA lysis buffer, all at 4° C. Cell pellets were then resuspended in 500 μl FA lysis buffer (plus protease inhibitors), transferred to a 2 ml microfuge tube containing 250 μl glass beads, and vortexed on highest setting for 30 min, 4° C. Lysates were pelleted at 14000 rpm for 15 min at 4° C., and the pellet containing the chromatin and cross-linked proteins was resuspended in 1 ml cold FA lysis buffer. To shear the chromatin, samples were sonicated on setting 5 for 4× 12 sec, with incubation on ice between pulses. Sheared lysates were then cleared by centrifugation at 14000 rpm, 30 min at 4° C. Typically, this yielded about 800 μl of cleared lysate, which was split in half and used for two independent IP reactions. To precipitate Rat1, 4 μl Ab116 (or 4 μl or pre-immune sera) and 10 μl ProA beads were incubated with 400 μl lysate, 90 min at 4° C. Beads were then washed in 700 μl of each of the following for 3 min at RT: twice with FA lysis buffer (150 mM NaCl); twice with FA lysis buffer (500 mM NaCl); ChIP wash buffer; 1× TE. Protein-chromatin complexes were then eluted off the beads in 100 μl ChIP elution buffer, 65° C., 10 min. Cross-linking was reversed by digestion with Pronase (2 mg/ml), 2 hr, 42° C. followed by 6 hr, 65° C. Chromatin was then purified using a QIAGEN PCR purification column and eluted in 100 μl Buffer EB (10 mM Tris). In FIG. 7A, association with the MAT α locus, comparing pre-immune and Ab116, as well as association with PMA1, demonstrated that Rat1 exhibited enhanced association with the MAT α locus only after induction of the break, and association was dependent on the Ab116 antibody. The results of three independent experiments, shown in FIG. 7B, demonstrated that Rat1 exhibited a reproducible and statistically significant, increase in association with the MAT α locus, with no accompanying increase in association to a control locus (PMA1) that was not adjacent to the break.

### Example 6

### Rat1 Localizes to Telomeres Preferentially During Late S Phase

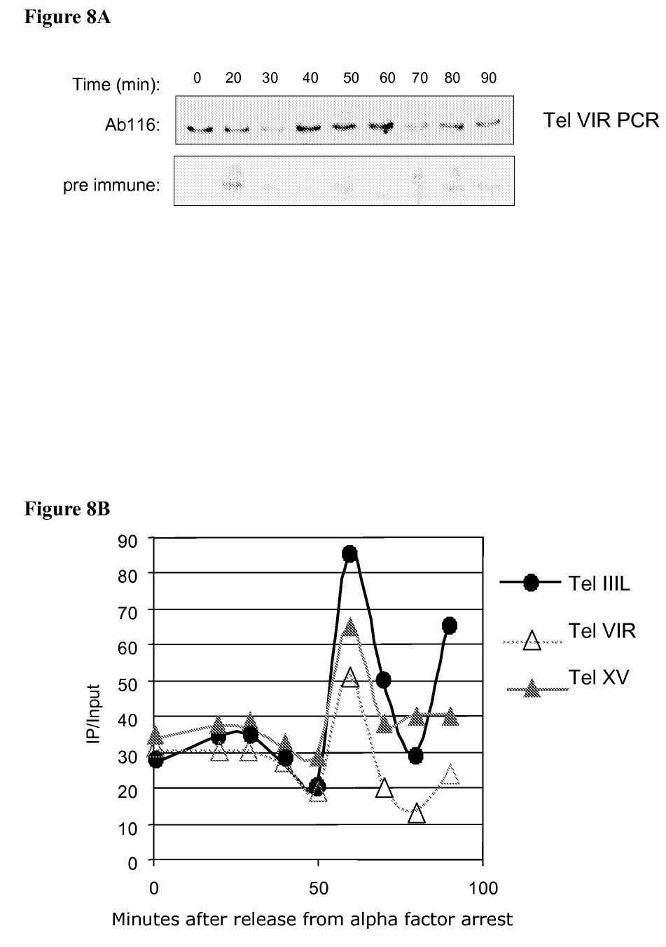

To examine whether Rat1 is associated with telomeres, a ChIP assay similar to that described in Example 5 was performed, on strains expressing either Rat1-TAP or Cdc13-myc18, from samples taken from synchronized cultures as cells progressed through the cell cycle. Cells were arrested with alpha factor and released into the cell cycle, using standard protocols for arrest and release, with samples taken every 10 minutes for up to 90 minutes. Position in the cell cycle was monitored by FACS analysis: 300 μl of EtOH-fixed cell suspension was rehydrated in 3 ml 50 mM NaCitrate, pelleted at RT, resuspended in 0.5 ml RNase A solution (2 mg/ml in 50 mM NaCitrate) and incubated at 37° C., 2-12 hr. 0.5 ml Sytox green solution (2 μM in 50 mM NaCitrate) was added to 1 μM final, samples were then stored at 4° C. in the dark. Just before FACS, samples were sonicated on setting 1 for 5 sec. The same ChIP protocol was used as in Example 5, except that Rat1-TAP was precipitated with 10 μl IgG Sepharose™ in FIG. 8B. Association of Rat1 with telomeric sequences in the immunoprecipitates was assessed by PCR using the Accuprime™ Taq PCR kit (Invitrogen) in the following 10 μl reaction: 1× Buffer I, 0.25 μM each primer, 1 0 ChIP sample, 0.5 μl AccuTaq, 0.1 mCi/ml [α-³²P]dCTP. PCR reactions were put through 25-30 cycles of 94° C., 15 sec; 42° C. 30 sec; 72° C., 1 min, and PCR reaction products were separated on native TBE 6% polyacrylamide gels, dried and detected on a phosphoimager. In FIG. 8B, three different telomeres were monitored, which demonstrates that Rat1 is present at telomeres throughout the cell cycle, but an increase in telomere association between 40 min and 60 min after alpha-factor release is observed, with all three sets of telomeric primers.

### Example 7

### Isolation of a Panel of Conditional Lethal Mutations in the S. Cerevisiae RAT1 Gene

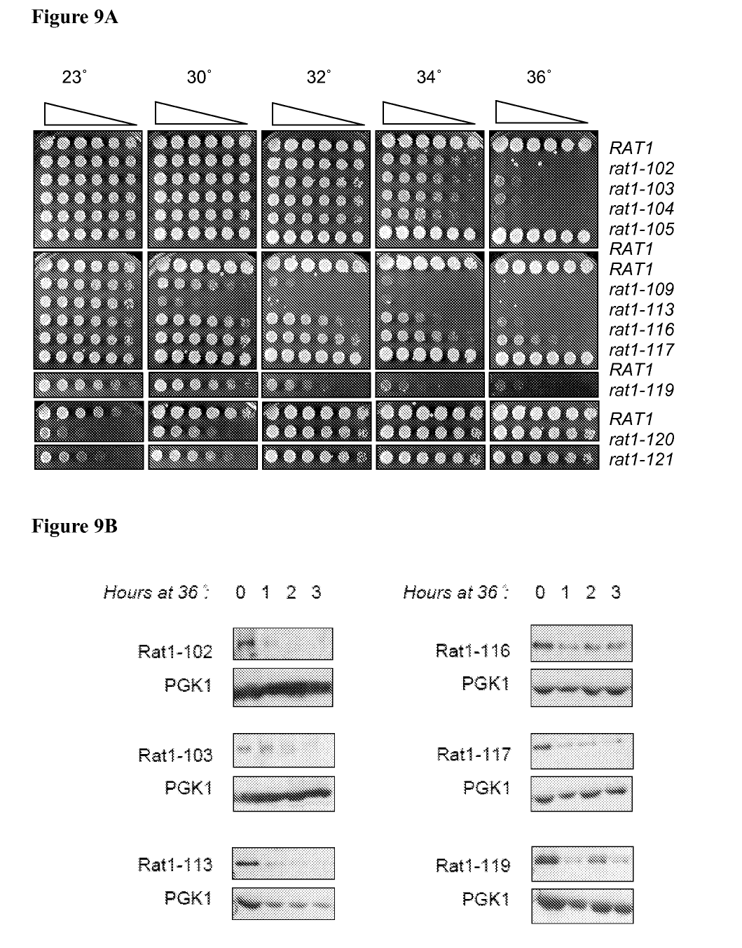

RAT1 is an essential gene, which does not permit manipulation of yeast cultures that lack the Rat1 protein, thereby precluding an analysis of telomere and double-strand breaks in the absence of Rat1 function. Therefore, a collection of temperature-sensitive alleles was isolated, which are viable at permissive temperatures but inviable at non-permissive temperatures. A library of mutagenized RAT1 alleles was generated from a plasmid bearing the RAT1 gene, expressed under the native RAT1 promoter (pAJ203; A. Johnson, Mol. Cell.Biol., 1997; 17: 6122-6130), using techniques that are standard (see Bertuch and Lundblad, Mol. Cell. Biol. 23, 8202-8215 2003), in order to generate approximately 1 nucleotide change per RAT1 gene per plasmid. pAJ203 was transformed into a mutagenic strain of E. coli (XL1 Red, Stratagene), to the manufacturer's protocol, to produce ˜32,000 colonies. This library of mutagenized plasmids was transformed into YVL3042 (MAT α rat1-Δ::KANMXura3-52 lys2-801 trp1-Δ1 his3-Δ200 leu2-Δ1/pVL3344 [URA3 CEN RAT1], which is an S. cerevisiae rat1-Δ shuffle strain kept alive by a covering wild type RAT1 plasmid; transformants were plated on complete media plates that lacked leucine (abbreviated as -Leu plates), in order to select for the newly introduced pAJ203 plasmid. Standard yeast transformation techniques were used. Transformation of YVL3042 with the mutagenized pool of pAJ203 plasmids generated ˜46,000 colonies, at a colony density of approximately 300 to 350 colonies per plate. Each -Leu plate was subsequently replica-plated onto two -Leu plates which also contained 5-FOA (5-fluoro-orotic acid), in order to select for spontaneous loss of the pVL3344 plasmid. Selection for spontaneous loss of URA3-containing plasmids by propagation on 5-FOA-containing yeast media is a standard yeast plasmid protocol (referred to as a “plasmid shuffle”). The two -Leu 5-FOA replicas were incubated at 23° or 36° C., and screened 24 hours later for re-growth of the replicas of each original colony. Isolates that failed to grow at 36° but grew at 23° were recovered, and the pAJ203 plasmid derivatives were isolated from each candidate strain that exhibited temperature-sensitive growth. Candidate plasmids were re-transformed into YVL3042 and re-examined for temperature-sensitive growth using this same protocol. This procedure resulted in 22 plasmids that exhibited temperature-sensitive growth in the absence of the endogenous RAT1 gene. The behavior of yeast strains expressing these rat1-ts mutations is shown in FIG. 9A which shows the growth of yeast strain derivatives of YVL3042, which have lost the pVL3344 plasmid but retain the derivatives of the pAJ203 plasmid expressing each of the different rat1-ts alleles. For each of these strains, 5-fold serial dilutions were plated on YPD media, which were subsequently incubated at the indicated temperature for 3 days and then photographed. Thermo-stability of the mutant Rat1 proteins was assessed for a subset of these Rat1-ts mutant proteins, using the anti-Rat1 peptide antibodies described in Example 4. The strains used for this analysis were the same as those used in FIG. 9A. Cultures were grown to mid-log phase (O.D.=0.4 to 0.6) in YPD media at room temperature, and shifted to 36° C. by the addition of pre-warmed YPD media and transfer to a shaking water bath; samples were flash frozen in liquid N₂, just prior to the temperature shift and at 1, 2 and 3 hours after incubation at 36° C. Whole cell extracts were prepared, and anti-Rat1 western analysis was performed, as described in Example 4; anti-PGK westerns were performed as previously described (Lee and Mandell, et. al. NSMB, 15:990-997 (2007). Results shown in FIG. 9B demonstrate that Rat1 protein is destabilized to a substantial degree in 5 of the 6 rat1-ts strains that were assayed for protein stability.

### Example 8

### Sequence Analysis of Amino Acid Changes in Conditional Lethal Mutations in the S. cerevisiae RAT1 Gene

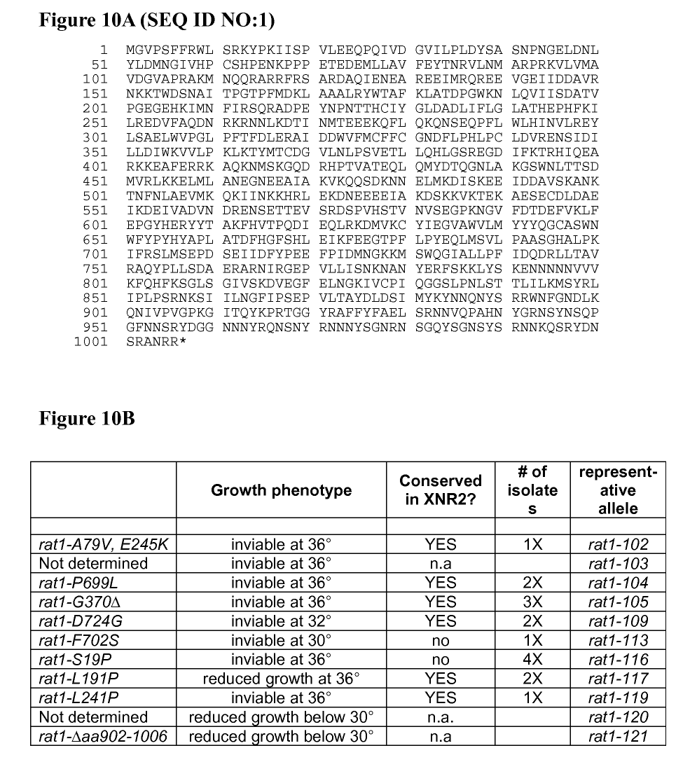

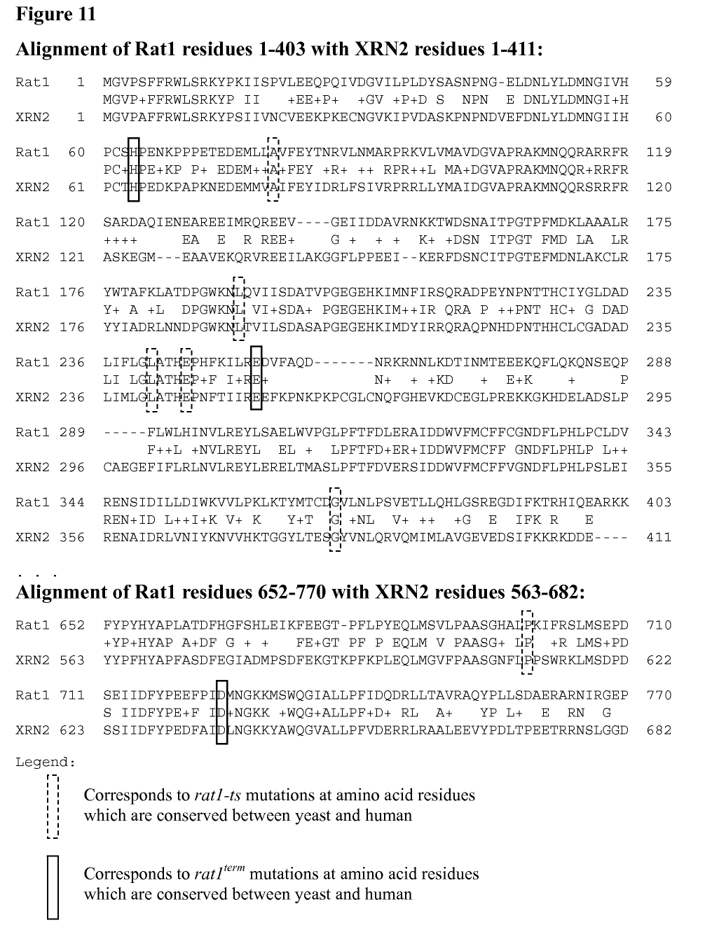

The RAT1 gene present on 19 of the plasmids rescued from the experiment described in Example 7 was sequenced, in order to detect mutations within the open reading frame of the RAT1 gene. The sequence of the wild type S. cerevisiae Rat1 protein is shown in FIG. 10A, and the results of the sequence analysis in FIG. 10B. Twelve of the mutant plasmids contained six different amino acid missense mutations in the Rat1 ORF: two of these missense mutations (F702A and L241P) were recovered only once, but the remaining four missense mutations were identified 2 to 4 times. Of the remaining 19 plasmids: one plasmid contained a double missense mutation (A79V E245K), one plasmid contained a 3 nucleotide deletion, resulting in an in-frame deletion of a single amino acid (AG370), a third plasmid resulted in a truncation of the Rat1 ORF, and for two plasmids the causative mutation was not identified. Notably, 6 of these alleles contained mutations in amino acid residues that were conserved between the yeast Rat1 and human XRN2 proteins. This is summarized in the Table shown in FIG. 10B, and illustrated in FIG. 11, which directly compares the S. cerevisiae Rat1 and H. sapiens XRN1 protein sequences. The residues that were identified in the rat1-ts mutant collection which are conserved between the yeast and human proteins are indicated in the figure.

### Example 9

### Isolation of Mutations in the S. Cerevisiae Rat1 Gene (Rat1^{Term}) which are Specifically Impaired for Terminase Activity

In order to recover mutations in RAT1 which specifically impair the terminase activity of Rat1 at telomeres and/or double-strand breaks (referred to as rat1^{term} alleles), a collection of site-directed mutations was constructed in charged residues predicted to be located on the predicted surface of the S. cerevisiae Rat1 protein. Identification of predicted surface residues was based on the published structure of the S. pombe Rat1 protein (Xiang et al. Nature 458:784-788 2009), which was used to construct a predicted structure for the S. cerevisiae protein. Charged residues were chosen for mutagenesis to an opposite charged residue (e.g. glutamic acid to arginine), on the assumption that a change in charge would have the potential to alter a protein-interaction site (for one example which supports this assumption, see Pennock et al. Cell 104(3):387-96 2001). A total of 132 amino acids, in the 1006 amino acid S. cerevisiae Rat1 protein, were mutated using standard site-directed mutagenesis protocols in pAJ203; sequence analysis across the location of the predicted mutation was used to confirm the mutational change. These 132 mutant plasmids were transformed into a panel of yeast strains (YVL3042 or derivatives of YVL3042), and examined for in vivo phenotypes that would be indicative of a defect in repair of double-strand breaks and/or impaired telomere function. The specific phenotypic tests that these 132 mutant plasmids were subjected to is as follows:

a. Viability in the rat1-Δ shuffle strain (YVL3042), following loss of the pVL3344 [URA3 CEN RAT1] plasmid was assessed. The 132 plasmids were transformed into YVL3042 and the resulting 132 strains were propagated on 5-FOA-containing media, to identify those mutations that were incapable of keeping the strain alive in the absence of the wild type RAT1 gene present on the pVL3344 plasmid. As would be expected for mutations that were restricted to the surface of the protein (and therefore unlikely to disrupt global protein structure), 129 strains were viable. The three mutations that conferred inviability were located in a pocket for nucleic acid binding, adjacent to the catalytic site. The 129 viable strains were further assessed for growth on rich media plates, at 23°, 30° and 36°, to determine whether any of these mutations exhibited conditional lethality. Only one mutation (rat1-D724K) displayed impaired growth at 36°, which is consistent with the fact that this residue was also identified in the rat1-ts screen described in Example 7 (rat1-109, which is due to a D→G mutation at residue 724).

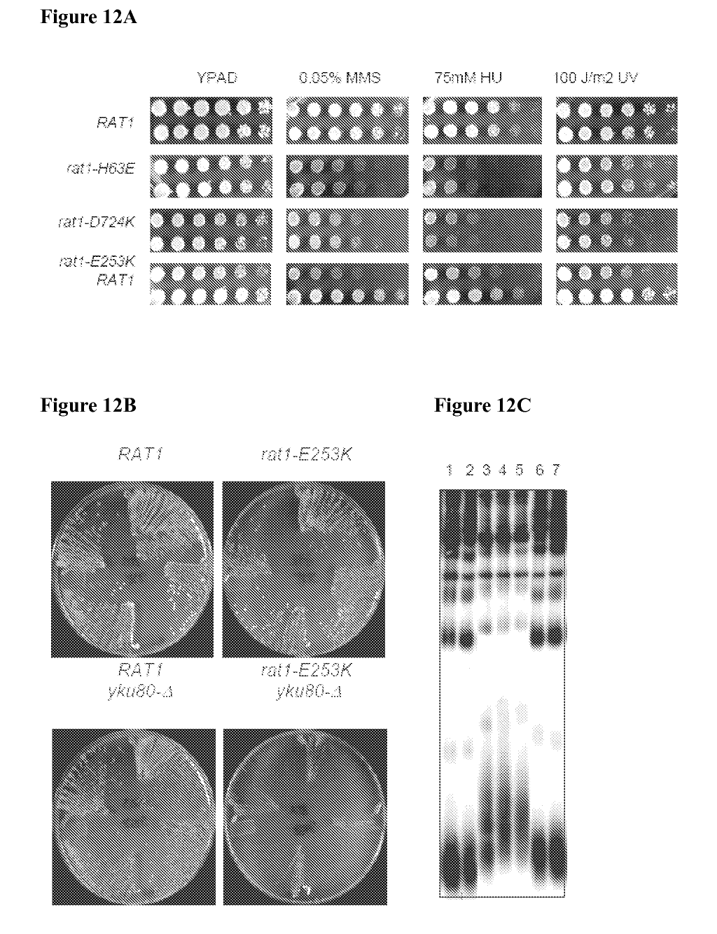

b. For those candidate plasmids expressing a viable allele of RAT1, as assessed in (a), strains expressing the 129 mutant alleles of RAT1 were examined for reduced viability in the presence of DNA damaging agents (100 J/m² ultraviolet light [UV], 0.05% methyl methane sulfate [MMS] and 75 mM hydroxyurea [UV]), using standard techniques (serial dilutions of cells were plated on media in the presence and absence of DNA damaging agents, and examined for growth after 72 hours). This identified three missense mutations in RAT1 (rat1-H63E, rat1-E253K and rat1-D724K) which exhibited enhanced sensitivity to HU and MMS, as well as a slight sensitivity to UV (FIG. 12A), indicating that strains expressing these three mutant alleles of RAT1 are impaired for their ability to repair induced DNA damage.

c. For those candidate plasmids expressing a viable allele of RAT1, as assessed in (a), strains expressing the 129 mutant alleles of RAT1 were examined for alterations in telomere length, using standard techniques. One mutation in RAT1 (rat1-D724K) resulted in a substantial elongation of telomeres, relative to wild type (FIG. 12B), indicating a defect in telomere length regulation.

d. For those candidate plasmids expressing a viable allele of RAT1, as assessed in (a), each of the 129 plasmids were introduced into four yeast strains, which were derivatives of YVL3042. Each of these four variants contained a genomic deletion of YKU80, MRE11, SAE2 or EXO1, respectively. The principle behind this phenotype test was that often defects in telomere function and/or defects in the ability to repair double-strand breaks can be synergistic, such that a defect in one gene-encoded activity may not be sufficiently severe enough to result in lethality, but a combination of defects confers non-viability (referred to as “synthetic lethality”); for example, see Nugent et al. Curr Biol. 8(11):657-60 1998. Each of the 129 plasmids encoding viable alleles of RAT1 were introduced into these four strains, and the resulting 516 strains were propagated on 5-FOA-containing media, to identify those mutations that were incapable of keeping the strain alive in the combined absence of a wild type RAT1 gene and either YKU80, MRE11, SAE2 or EXO1 function, respectively. This synthetic lethality assay identified one mutation, rat1-E253K, which displayed a severe synthetic lethality in the absence of YKU80 function (FIG. 12C): a rat1-E253K YKU80 strain was viable (although sensitive to DNA damage, FIG. 12A), whereas a rat1-E253K yku80-Δ strain was unviable. The degree of synthetic lethality displayed by this double mutant combination was comparable to that observed previously when a defect in telomerase was combined with a yku80-Δ mutation (Nugent et al. Curr Biol. 8(11):657-60 1998).

These in vivo phenotypic characterizations demonstrate that three rat1^{term} mutations have been identified, which specifically impair the ability of the Rat1 to perform its terminase activity at telomeres and/or double-strand breaks. These three rat1^{term} mutations are located in three amino acid residues which are completely conserved between the yeast Rat1 and human XRN2 proteins, as displayed in the sequence alignment shown in FIG. 11 (the three rat1^{term} residues are indicated by solid boxes), indicating that these terminase-specific alleles isolated in the yeast protein have identified a highly conserved function of the Rat1/XRN2 protein.

### Example 10

### Depletion of Rat1 Function Impairs Resection of an Experimentally Induced Double-Strand Break

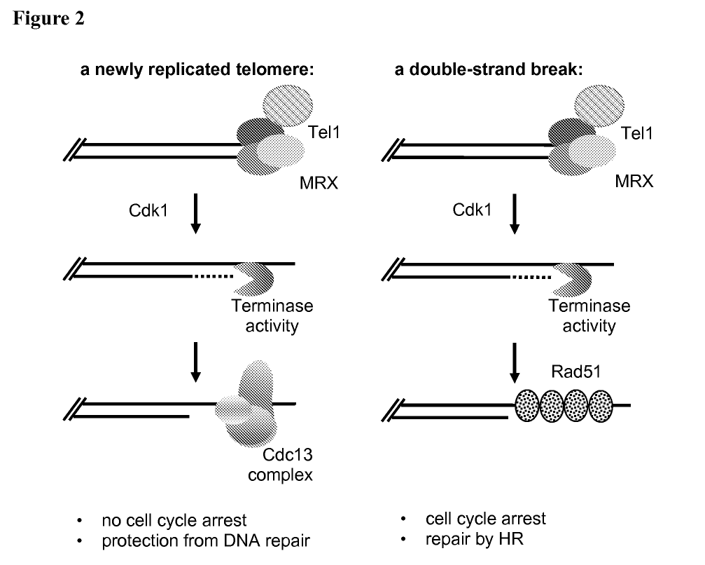

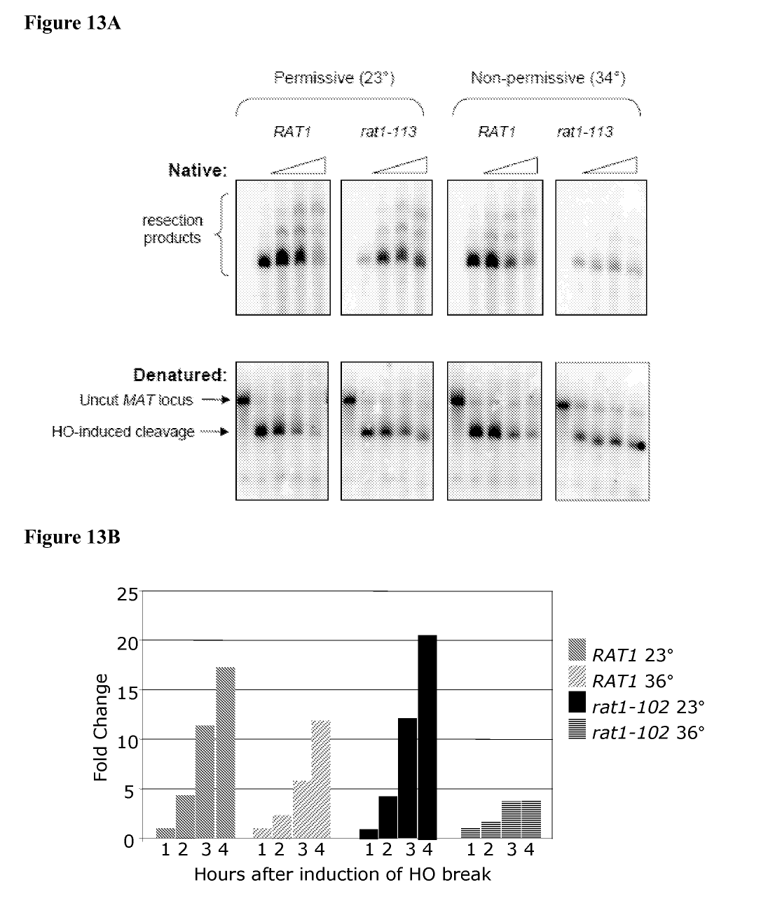

RAT1 and rat1-113 cultures were grown to mid-log in raffinose media, incubated in nocodozole to arrest cells in G2/M, incubated for one hour at permissive or non-permissive temperatures, to either retain or inactivate Rat1-113 function, and finally incubated in galactose media to induce cleavage of the HO site at the MAT locus. Genomic DNA was isolated from samples taken at 0, 1, 2, 3 and 4 hours, hybridized with a ³²P-labeled single-stranded oligo designed to detect resection on one side of the HO break and resolved on a 0.7% agarose gel under native conditions. The native gel was subsequently denatured, and re-probed, to monitor the efficiency of HO cleavage, which was equivalent in all samples. As shown in FIG. 13A, the extent of resection in the rat1-113 strain was dramatically reduced, as indicated by two criteria: (i) the reduced signal on the native gel for the major resection product and (ii) the absence of the higher molecular weight resection products that appear in later timepoints in the wild type samples (which correspond to loss of sites for the restriction enzyme EcoRI, as the nuclease degrades inward from the HO-generated double-strand break; EcoRI will only digest duplex DNA and not single-stranded DNA). FIG. 13B shows the results of a ChIP experiment, monitoring association of the Rad51 protein with single-stranded DNA following cleavage by the HO nuclease (see FIG. 2). As expected from prior published studies, Rad51 exhibits extensive association, in a break-dependent manner, providing a sensitive assay for the appearance of newly exposed single-stranded DNA (FIG. 13B). In a rat1-102 strain at non-permissive temperatures, however, association of Rad51 with the newly generated DSB is greatly reduced at non-permissive temperatures, consistent with the conclusion that the terminase activity of the Rat1 protein is required in order to degrade a newly generated DSB.

### Example 11

### Lethality Due to Depletion of Rat1 can be Partially Rescued by lig4-Δ

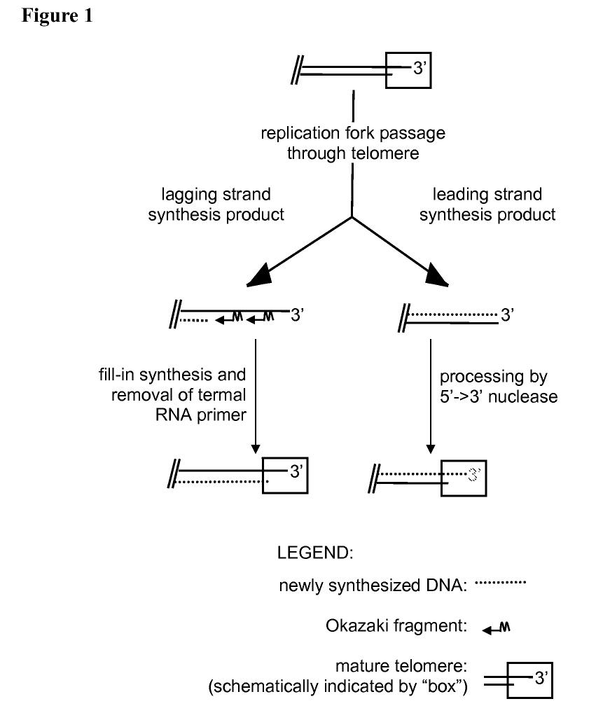

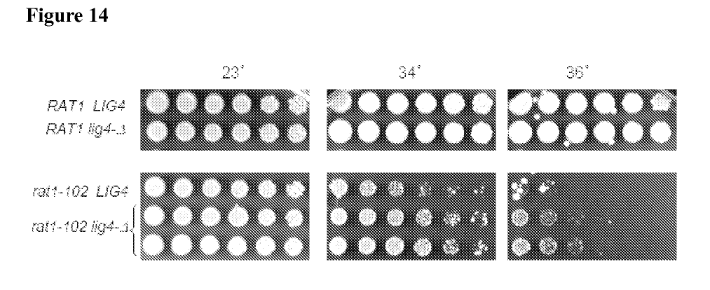

The model presented in FIGS. 1 and 2 argues that in the absence of the terminase activity of the Rat1 protein at telomeres, the resulting blunt chromosome termini should become subject to end-to-end fusions. Such chromosomal fusions should be a lethal event for cells. Under conditions in which Rat1 function is partially, but not completely, impaired, it is likely that only a subset of chromosome termini will remain completely blunt-ended and thus substrates for fusion. If fusion could be prevented by removal of the Lig4 ligase, such chromosomes might be able to progress through the cell cycle and become adequately processed in the next cell cycle. To test this, a panel of rat1-ts mutations were examined for viability in LIG4 and lig4-Δ strain backgrounds, by plating serial dilutions at a range of temperatures on YPD plates; as shown in FIG. 14, the lethality of the rat1-102 strain can be partially relieved by the loss of Lig4 function.

### Example 12

### The Rat1 Terminase Protein is in a Complex with the Telomerase RNP

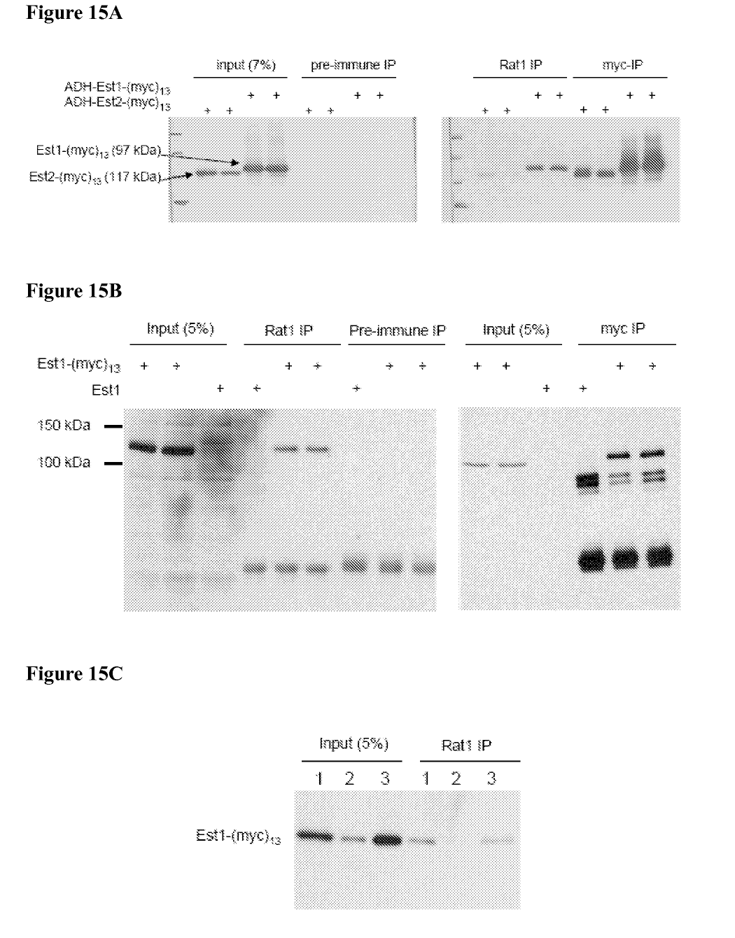

Co-immunoprecipitation experiments were performed to examine whether the Rat1 protein and telomerase physically associated with each other while performing their two enzymatic activities at telomeres. In FIG. 15A, extracts were prepared from two strains containing high copy plasmids expressing either Est1-(myc)13 or Est2-(myc)13, and subject to immunoprecipitation with either anti-Rat1 peptide antibodies or pre-immune sera, using standard IP protocols. Association between Est1 and Rat1 could be observed, at ˜5% efficiency; the association was specific, as no interaction could be observed using the pre-immune sera (FIG. 15A). A weaker association was also observed between Est2 and Rat1; however, the myc tag introduced into the C-terminus of the Est2 protein appeared to be degraded off, making this a less useful reagent. Subsequent experiments monitored the association of Rat1 with endogenous levels of the Est1 protein, with the (myc)13 tag introduced into the genome, at the C-terminus of the Est1 ORF. As shown in FIG. 15B, an association between Est1 and Rat1 could still be observed, although somewhat reduced relative to the over-expression situation shown in FIG. 15A. The formation of a complex between Rat1 and the Est1 telomerase subunit appeared to be cell cycle regulated, as assessed by comparing co-immunoprecipitation between Est1 and Rat1 from cultures that were either asynchronous, or arrested in G1 phase with alpha-factor, or arrested in G2/M with nocodazole. The degree of association between Est1 and Rat1 was reduce in both of the arrested samples, as compared to the asynchronous samples (FIG. 15C), consistent with the observations in Example 6 that indicates that the peak of Rat1 association with telomeres is highest in late S phase.

### Informal Sequence Listings

It is understood that the terms “locus,” “version,” “GI” and like in the context of sequences listings refer to terms customarily employed in the art and by the National Center for Biotechnology Information (NCBI).

Sequence of Rat1 (SEQ ID NO:1), NCBI locus NP_{—}014691, version NP_{—}014691.1, GI:6324622:

```
    1 MGVPSFFRWL SRKYPKIISP VLEEQPQIVD GVILPLDYSA SNPNGELDNL YLDMNGIVHP
  61 CSHPENKPPP ETEDEMLLAV FEYTNRVLNM ARPRKVLVMA VDGVAPRAKM NQQRARRFRS
121 ARDAQIENEA REEIMRQREE VGEIIDDAVR NKKTWDSNAI TPGTPFMDKL AAALRYWTAF
181 KLATDPGWKN LQVIISDATV PGEGEHKIMN FIRSQRADPE YNPNTTHCIY GLDADLIFLG
241 LATHEPHFKI LREDVFAQDN RKRNNLKDTI NMTEEEKQFL QKQNSEQPFL WLHINVLREY
301 LSAELWVPGL PFTFDLERAI DDWVFMCFFC GNDFLPHLPC LDVRENSIDI LLDIWKVVLP
361 KLKTYMTCDG VLNLPSVETL LQHLGSREGD IFKTRHIQEA RKKEAFERRK AQKNMSKGQD
421 RHPTVATEQL QMYDTQGNLA KGSWNLTTSD MVRLKKELML ANEGNEEAIA KVKQQSDKNN
481 ELMKDISKEE IDDAVSKANK TNFNLAEVMK QKIINKKHRL EKDNEEEEIA KDSKKVKTEK
541 AESECDLDAE IKDEIVADVN DRENSETTEV SRDSPVHSTV NVSEGPKNGV FDTDEFVKLF
601 EPGYHERYYT AKFHVTPQDI EQLRKDMVKC YIEGVAWVLM YYYQGCASWN WFYPYHYAPL
661 ATDFHGFSHL EIKFEEGTPF LPYEQLMSVL PAASGHALPK IFRSLMSEPD SEIIDFYPEE
721 FPIDMNGKKM SWQGIALLPF IDQDRLLTAV RAQYPLLSDA ERARNIRGEP VLLISNKNAN
781 YERFSKKLYS KENNNNNVVV KFQHFKSGLS GIVSKDVEGF ELNGKIVCPI QGGSLPNLST
841 TLILKMSYRL IPLPSRNKSI ILNGFIPSEP VLTAYDLDSI MYKYNNQNYS RRWNFGNDLK
901 QNIVPVGPKG ITQYKPRTGG YRAFFYFAEL SRNNVQPAHN YGRNSYNSQP GFNNSRYDGG
961 NNNYRQNSNY RNNNYSGNRN SGQYSGNSYS RNNKQSRYDN SRANRR
```

Sequence of XRN2 (SEQ ID NO:2), NCBI locus NP_{—}036387, version NP_{—}036387.2, GI:18860916:

```
    1 MGVPAFFRWL SRKYPSIIVN CVEEKPKECN GVKIPVDASK PNPNDVEFDN LYLDMNGIIH
  61 PCTHPEDKPA PKNEDEMMVA IFEYIDRLFS IVRPRRLLYM AIDGVAPRAK MNQQRSRRFR
121 ASKEGMEAAV EKQRVREEIL AKGGFLPPEE IKERFDSNCI TPGTEFMDNL AKCLRYYIAD
181 RLNNDPGWKN LTVILSDASA PGEGEHKIMD YIRRQRAQPN HDPNTHHCLC GADADLIMLG
241 LATHEPNFTI IREEFKPNKP KPCGLCNQFG HEVKDCEGLP REKKGKHDEL ADSLPCAEGE
301 FIFLRLNVLR EYLERELTMA SLPFTFDVER SIDDWVFMCF FVGNDFLPHL PSLEIRENAI
361 DRLVNIYKNV VHKTGGYLTE SGYVNLQRVQ MIMLAVGEVE DSIFKKRKDD EDSFRRRQKE
421 KRKRMKRDQP AFTPSGILTP HALGSRNSPG SQVASNPRQA AYEMRMQNNS SPSISPNTSF
481 TSDGSPSPLG GIKRKAEDSD SEPEPEDNVR LWEAGWKQRY YKNKFDVDAA DEKFRRKVVQ
541 SYVEGLCWVL RYYYQGCASW KWYYPFHYAP FASDFEGIAD MPSDFEKGTK PFKPLEQLMG
601 VFPAASGNFL PPSWRKLMSD PDSSIIDFYP EDFAIDLNGK KYAWQGVALL PFVDERRLRA
661 ALEEVYPDLT PEETRRNSLG GDVLFVGKHH PLHDFILELY QTGSTEPVEV PPELCHGIQG
721 KFSLDEEAIL PDQIVCSPVP MLRDLTQNTV VSINFKDPQF AEDYIFKAVM LPGARKPAAV
781 LKPSDWEKSS NGRQWKPQLG FNRDRRPVHL DQAAFRTLGH VMPRGSGTGI YSNAAPPPVT
841 YQGNLYRPLL RGQAQIPKLM SNMRPQDSWR GPPPLFQQQR FDRGVGAEPL LPWNRMLQTQ
901 NAAFQPNQYQ MLAGPGGYPP RRDDRGGRQG YPREGRKYPL PPPSGRYNWN
```

Sequence of synthetic DNA oligomer described in Example 3:

(SEQ ID NO: 3)
5′-CCTCTAGGTTCCTTTGTTAC-3′.

Sequence of synthetic DNA oligomer described in Example 3:

(SEQ ID NO: 4)
5′-GTAACAAAGGAACCTAGAGG-3′

Sequence of anti-peptide antibody described in Example 4:

(SEQ ID NO: 5)
YSGNSYSRNNKQSRYDNSRANRR.

Sequence of anti-peptide antibody described in Example 4:

(SEQ ID NO: 6)
NDRENSETTEVSRD.

Sequence of synthetic DNA primer for MAT α locus described in Example 5:

(SEQ ID NO: 7)
5′-AGATGTGTTTGTACATTTGGCCTTATA-3′

Sequence of synthetic DNA primer for MAT α locus described in Example 5:

(SEQ ID NO: 8)
5′-TACCCTTATCTACTTGCCTCTTTTGTT-3′.

Sequence of synthetic DNA primer for PMA1 locus described in Example 5:

(SEQ ID NO: 9)
5′-ATGAACCACAGTACATCATATTAAGACGTA-3.′

Sequence of synthetic DNA primer for PMA1 locus described in Example 5:

(SEQ ID NO: 10)
5′-GAAGAAATTTCGATGCGATTAACC-3.

What is claimed is:

1. A cell comprising a nucleic acid encoding a terminase protein with an impaired terminase activity.

2. The cell of claim 1, wherein said cell is a yeast cell.

3. The cell of claim 1, wherein said cell is a mammalian cell.

4. The cell of claim 1, wherein said cell is a human cell.

5. The cell of claim 1, wherein said cell is a recombinant cell.

6. The cell of claim 1, wherein said terminase protein is a recombinant terminase protein.

7. The cell of claim 1, further comprising a recombinant nucleic acid.

8. The cell of claim 1, wherein said impaired terminase activity is increased when compared to a terminase activity of a control terminase protein.

9. The cell of claim 1, wherein said impaired terminase activity is decreased when compared to a terminase activity of a control terminase protein.

10. A method for preparing a cell expressing a terminase protein with an impaired terminase activity comprising:
(i) introducing to said cell a nucleic acid encoding said terminase protein with said impaired terminase activity,
(ii) allowing said cell to express said terminase protein with said impaired terminase activity; and
(iii) comparing said impaired terminase activity in said cell to a terminase activity in a control cell.

11. The method of claim 10, wherein said impaired terminase activity is increased when compared to said terminase activity of said control cell.

12. The method of claim 10, wherein said impaired activity is decreased when compared to said terminase activity of said control cell.

13. A method of determining whether a test compound modulates terminase activity in vitro, said method comprising:
(i) combining a test compound, a terminase protein, and a double-stranded deoxynucleotide substrate in a single reaction mixture in vitro under conditions conducive to terminase activity;
(ii) allowing sufficient time for said terminase protein to react with said double-stranded deoxynucleotide substrate to form a hydrolyzed double-stranded deoxynucleotide product; and
(iii) detecting an amount of said hydrolyzed double-stranded deoxynucleotide product and comparing said amount to a control amount of hydrolyzed double-stranded deoxynucleotide product, thereby determining whether said test compound modulates terminase activity in vitro.

14. The method of claim 13 wherein said double-stranded deoxynucleotide substrate is a blunt ended double-stranded deoxynucleotide substrate.

15. The method of claim 14, wherein said hydrolyzed double-stranded deoxynucleotide product is a 3′-overhang double-stranded deoxynucleotide product.

16. A method of determining whether a test compound modulates terminase activity in a cell, said method comprising:
(i) determining whether said test compound modulates terminase activity in vitro according to the method of claim 13;
(ii) contacting said test compound with a cell; and
(iii) determining an amount of telomeric stability in said cell and comparing said amount to a control amount of telomeric stability, thereby determining whether said test compound modulates terminase activity in said cell.

17. A method of determining whether a test compound is a terminase modulating anticancer compound, the method comprising:
(i) determining whether a test compound modulates terminase activity in vitro according to the method of claim 13;
(ii) administering said test compound to a cancer model organism; and
(iii) determining whether said test compound exhibits anticancer properties in said cancer model organism.

18. The method of claim 17, wherein said method further comprises after step (i) and before step (ii),
(a) contacting said test compound with a cell; and
(b) determining an amount of telomeric stability in said cell and comparing said amount to a control amount of telomeric stability, thereby determining whether said test compound modulates telomeric terminase activity in said cell.

19. A method of determining whether a test compound modulates terminase activity at double-strand breaks in a cell, the method comprising:
(i) determining whether said test compound modulates terminase activity in vitro according to the method of claim 13;
(ii) contacting said test compound with a cell; and
(iii) determining an amount of DNA repair in said cell and comparing said amount to a control amount of DNA repair in said cell thereby determining whether said test compound modulates DNA repair of a double-strand break of a double-stranded nucleic acid in said cell by modulating terminase activity.

20. A kit for determining whether a test compound modulates terminase activity in vitro, said kit comprising a terminase protein and a double-stranded deoxynucleotide substrate.


SEQUENCE LISTING (10 sequences)

SEQ ID NO: 1 - protein, 1006 aa, Saccharomyces cerevisiae
MGVPSFFRWLSRKYPKIISPVLEEQPQIVDGVILPLDYSASNPNGELDNLYLDMNGIVHPCSHPENKPPPETEDEMLLAVFEYTNRVLNMARPRKVLVMAVDGVAPRAKMNQQRARRFRSARDAQIENEAREEIMRQREEVGEIIDDAVRNKKTWDSNAITPGTPFMDKLAAALRYWTAFKLATDPGWKNLQVIISDATVPGEGEHKIMNFIRSQRADPEYNPNTTHCIYGLDADLIFLGLATHEPHFKILREDVFAQDNRKRNNLKDTINMTEEEKQFLQKQNSEQPFLWLHINVLREYLSAELWVPGLPFTFDLERAIDDWVFMCFFCGNDFLPHLPCLDVRENSIDILLDIWKVVLPKLKTYMTCDGVLNLPSVETLLQHLGSREGDIFKTRHIQEARKKEAFERRKAQKNMSKGQDRHPTVATEQLQMYDTQGNLAKGSWNLTTSDMVRLKKELMLANEGNEEAIAKVKQQSDKNNELMKDISKEEIDDAVSKANKTNFNLAEVMKQKIINKKHRLEKDNEEEEIAKDSKKVKTEKAESECDLDAEIKDEIVADVNDRENSETTEVSRDSPVHSTVNVSEGPKNGVFDTDEFVKLFEPGYHERYYTAKFHVTPQDIEQLRKDMVKCYIEGVAWVLMYYYQGCASWNWFYPYHYAPLATDFHGFSHLEIKFEEGTPFLPYEQLMSVLPAASGHALPKIFRSLMSEPDSEIIDFYPEEFPIDMNGKKMSWQGIALLPFIDQDRLLTAVRAQYPLLSDAERARNIRGEPVLLISNKNANYERFSKKLYSKENNNNNVVVKFQHFKSGLSGIVSKDVEGFELNGKIVCPIQGGSLPNLSTTLILKMSYRLIPLPSRNKSIILNGFIPSEPVLTAYDLDSIMYKYNNQNYSRRWNFGNDLKQNIVPVGPKGITQYKPRTGGYRAFFYFAELSRNNVQPAHNYGRNSYNSQPGFNNSRYDGGNNNYRQNSNYRNNNYSGNRNSGQYSGNSYSRNNKQSRYDNSRANRR

SEQ ID NO: 2 - protein, 950 aa, Homo sapiens
MGVPAFFRWLSRKYPSIIVNCVEEKPKECNGVKIPVDASKPNPNDVEFDNLYLDMNGIIHPCTHPEDKPAPKNEDEMMVAIFEYIDRLFSIVRPRRLLYMAIDGVAPRAKMNQQRSRRFRASKEGMEAAVEKQRVREEILAKGGFLPPEEIKERFDSNCITPGTEFMDNLAKCLRYYIADRLNNDPGWKNLTVILSDASAPGEGEHKIMDYIRRQRAQPNHDPNTHHCLCGADADLIMLGLATHEPNFTIIREEFKPNKPKPCGLCNQFGHEVKDCEGLPREKKGKHDELADSLPCAEGEFIFLRLNVLREYLERELTMASLPFTFDVERSIDDWVFMCFFVGNDFLPHLPSLEIRENAIDRLVNIYKNVVHKTGGYLTESGYVNLQRVQMIMLAVGEVEDSIFKKRKDDEDSFRRRQKEKRKRMKRDQPAFTPSGILTPHALGSRNSPGSQVASNPRQAAYEMRMQNNSSPSISPNTSFTSDGSPSPLGGIKRKAEDSDSEPEPEDNVRLWEAGWKQRYYKNKFDVDAADEKFRRKVVQSYVEGLCWVLRYYYQGCASWKWYYPFHYAPFASDFEGIADMPSDFEKGTKPFKPLEQLMGVFPAASGNFLPPSWRKLMSDPDSSIIDFYPEDFAIDLNGKKYAWQGVALLPFVDERRLRAALEEVYPDLTPEETRRNSLGGDVLFVGKHHPLHDFILELYQTGSTEPVEVPPELCHGIQGKFSLDEEAILPDQIVCSPVPMLRDLTQNTVVSINFKDPQFAEDYIFKAVMLPGARKPAAVLKPSDWEKSSNGRQWKPQLGFNRDRRPVHLDQAAFRTLGHVMPRGSGTGIYSNAAPPPVTYQGNLYRPLLRGQAQIPKLMSNMRPQDSWRGPPPLFQQQRFDRGVGAEPLLPWNRMLQTQNAAFQPNQYQMLAGPGGYPPRRDDRGGRQGYPREGRKYPLPPPSGRYNWN

SEQ ID NO: 3 - DNA, 20 bp, Artificial
  Synthetic DNA oligonucleotide for detecting specific DNA exonuclease activity
cctctaggttcctttgttac

SEQ ID NO: 4 - DNA, 20 bp, Artificial
  Synthetic DNA oligonucleotide for detecting specific DNA exonuclease activity
gtaacaaaggaacctagagg

SEQ ID NO: 5 - protein, 23 aa, Artificial
  Synthetic peptide for use as anti-peptide antibody
YSGNSYSRNNKQSRYDNSRANRR

SEQ ID NO: 6 - protein, 14 aa, Artificial
  Synthetic peptide for use as anti-peptide antibody
NDRENSETTEVSRD

SEQ ID NO: 7 - DNA, 27 bp, Artificial
  Synthetic DNA primer for MAT alpha locus
agatgtgtttgtacatttggccttata

SEQ ID NO: 8 - DNA, 27 bp, Artificial
  Synthetic DNA primer for MAT alpha locus
tacccttatctacttgcctcttttgtt

SEQ ID NO: 9 - DNA, 30 bp, Artificial
  Synthetic DNA primer for PMA1 locus
atgaaccacagtacatcatattaagacgta

SEQ ID NO: 10 - DNA, 24 bp, Artificial
  Synthetic DNA primer for PMA1 locus
gaagaaatttcgatgcgattaacc